SEQUENCE LISTING (3 sequences)

SEQ ID NO: 1 - protein, 524 aa, Homo sapiens
  MISC_FEATURE (1)..(524): Human tissue nonspecific alkaline phosphatase (ALPL)
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 2 - protein, 533 aa, Bos taurus
  MISC_FEATURE (1)..(533): Bovine intestine alkaline phosphatase (CIP)
MQGACVLLLLGLHLQLSLGLVPVEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCKTTRGNEVTSVMNRAKKAGKSVGVVTTTRVQHASPAGAYAHTVNRNWYSDADLPADAQMNGCQDIAAQLVNNMDIDVILGGGRKYMFPVGTPDPEYPDDASVNGVRKRKQNLVQAWQAKHQGAQYVWNRTALLQAADDSSVTHLMGLFEPADMKYNVQQDHTKDPTLQEMTEVALRVVSRNPRGFYLFVEGGRIDHGHHDDKAYMALTEAGMFDNAIAKANELTSELDTLILVTADHSHVFSFGGYTLRGTSIFGLAPSKALDSKSYTSILYGNGPGYALGGGSRPDVNDSTSEDPSYQQQAAVPQASETHGGEDVAVFARGPQAHLVHGVEEETFVAHIMAFAGCVEPYTDCNLPAPTTATSIPDAAHLAASPPPLALLAGAMLLLLAPTLY

SEQ ID NO: 3 - protein, 404 aa, Pandalus borealis
  MISC_FEATURE (1)..(404): Shrimp alkaline phosphatase (rSAP)
MIGRTTFIALFVKVLTIWSFTKGEDCVWDNDVDYPEYPPLILDSSFQLVLPVLEGDQRITSVQSGSELILACPGREISALGSEDAQATCLGGKLVEVDGKEWNIVELGCTKMASETIHRNLGQCGDQDLGIYEVIGFDLPTTGHFYELIRVCFDPANETTIFSENIVHGASIAAKDIDPGRPSFKTSTGFFSVSMISVYSQRNQLELMKNLLGDDELAATIIDPSKQFYFAKGHMAPDADFVTVVEQDATYYYINALPQWQAFNNGNWKYLEYDTRDLAEKHGTDLTVYSGGWGVLELEDINGNPVEIYLGLAQDKKVVPAPALTWKVIYEKDTNRAAAIVGINNPHITTAPEPLCTDICSSLTWLDFDFGDLVHGYTYCCSVADLRAAIPNVPDLGDVDILDE

(19) United States
(12) Patent Application Publication
(10) Pub. No.: US 2023/0220346 A1
(43) Pub. Date: Jul. 13, 2023

(54) BIOLOGICALLY SYNTHESIZED HYDROXYAPATITE FOR BONE REGENERATION AND TISSUE ENGINEERING

(71) Applicant: BIOVENTURES, LLC, Little Rock, AR (US)

(72) Inventors: Erming Tian, Little Rock, AR (US); Maurizio Zangari, Little Rock, AR (US)

(21) Appl. No.: 17/769,663

(22) PCT Filed: Oct. 16, 2020
(86) PCT No.: PCT/US2020/055921

§ 371 Date: Apr. 15, 2022

Related U.S. Application Data
(60) Provisional application No. 62/915,843, filed on Oct. 16, 2019.

(51) Int. Cl. C12N 5/077; C12Q 1/42; C12Q 1/26

(52) U.S. Cl. CPC C12N 5/0654; C12Q 1/42; C12Q 1/26; C12N 2500/14; C12N 2501/73; C12N 2500/42

(57) ABSTRACT

Herein the inventors demonstrate that mineralization is a natural ability of cells cultured with at least two elements: calcium and acyclic alkane phosphoester salt or inorganic phosphate salt. The present invention provides methods for producing hydroxyapatite (HAP) in cell culture by supplying cells with these elements. The natural HAP crystals produced by these methods may be utilized in biomedical applications such as bone grafting. Also provided are methods of measuring organic phosphates in a sample from a subject and methods of measuring the glycerophosphates in a sample from a subject.

### CROSS-REFERENCE TO RELATED APPLICATIONS

This patent application claims the benefit of priority of United States Provisional Patent Application No. 62/915,843, filed Oct. 16, 2019, which is incorporated herein by reference in its entirety.

### SEQUENCE LISTING

A Sequence Listing accompanies this application and is submitted as an ASCII text file of the sequence listing named “169852_00075_ST25.txt” which is 12.9 KB in size and was created on Oct. 9, 2020. The sequence listing is electronically submitted via EFS-Web with the application and is incorporated herein by reference in its entirety.

### INTRODUCTION

Bone provides the framework for the body, protects the vital organs, supports mechanical movement, hosts hematopoietic cells, and maintains iron homeostasis. Bone remodeling is a lifelong process in which mature bone is removed from the skeleton through osteoclastic resorption and new bone is formed through osteoblastic formation. To achieve homeostasis, these remodeling processes are tightly regulated by a wide variety of signaling pathways [1,2] that couple bone resorption by osteoclasts [3-5] and bone formation by osteoblasts [6-10], and imbalances in these mechanisms result in bone diseases [11].

Osteoblasts are specialized cells that synthesize bone. For centuries, osteoblasts were thought to be the only cells able to perform this essential function. Groups of osteoblasts use calcium and inorganic phosphorus to produce the crystalline mineral hydroxyapatite (HAP). These calcium phosphate nanocrystals are deposited in a collagen matrix to harden bones in a process known as biomineralization [12,13]. The induction of osteoblast differentiation was previously considered the essential first step of biomineralization [14-16]. However, both the morphology and gene expression profile of osteoblasts are similar to those of fibroblasts, and there is no evidence suggesting that biomineralization is orchestrated by specific genes expressed in osteoblasts [17-19]. In this context, osteoblasts can be viewed as sophisticated fibroblasts, which can be identified by measuring a mineralized extracellular matrix when the cells are exposed to an environment containing calcium ions, β-glycerophosphate, ascorbic acid, dexamethasone, and serum (fetal bovine serum, FBS) for a period of 3-4 weeks [20,21].

While the regenerative properties of bone allow the vast majority of bone defects to heal spontaneously under suitable physiological conditions, the healing process is slow. Further, defects that are more substantial may not heal spontaneously for several reasons. Malignant bone lesions, for example, pose a considerable medical challenge. Cancers such as multiple myeloma and breast cancer frequently result in osteolytic lesions, which cause pain, spinal cord compression, and increase the risk of bone fracture and mortality. At diagnosis, over 80% of patients with multiple myeloma and up to 70% of patients with breast cancer have bone lesions caused by metastasis of cancer cells to the skeletal system. In myeloma patients, these bone lesions rarely heal, even in patients that have achieved complete cancer remission. Overall, the median survival after a bone metastasis diagnosis is only 19-25 months. Current treatment options, such as bisphosphonates or radiation, rarely cure these lesions [22,23].

Bone grafts are commonly used to fill bone defects. Although autograft bone (i.e., bone from that patient's own body) is considered the current gold standard, it is limited in supply and can result in donor site pain or hemorrhage. Lack of understanding of the biomineralization mechanism in vivo has also hindered implementation. Allografts, on the other hand, pose the risk of immune-mediated rejection and transmission of infectious diseases. To overcome these limitations, there is increasing interest in natural and biomimetic bone substitutes.

HAP is widely used as an implant material due to its excellent osteoconductive properties. This mineral has been used for varied applications, including skeletal reconstruction, dental implants, and nanoparticle targeted therapies. HAP is synthesized for such purposes by several traditional methods, including precipitation techniques, sol-gel approaches, hydrothermal techniques, multiple emulsion techniques, biomimetic deposition techniques, and electrodeposition techniques [24]. The most commonly used methods are known as ‘wet chemical’ techniques, which involve precipitation of HAP from an aqueous solution containing calcium and phosphate precursors. One wet chemical technique, for example, utilizes a slow precipitation reaction, in which a solution of orthophosphoric acid is added in a dropwise manner to a dilute solution of calcium hydroxide at a temperature of about 90° C.

The quality of synthesized HAP is determined by its homogeneity and porosity. One problem with the wet chemical technique is that the resulting HAP may contain voids, which is deleterious to its mechanical strength. To remove the voids, an additional densification step is often required. Another problem with the wet chemical technique is that the unreacted calcium and phosphate precursors in the precipitation reaction generate impurity phases. This results in formation of HAP that is non-homogeneous and lacking in crystallinity. Further, in precipitation reactions, the particles tend to agglomerate, making it difficult to control the size of the particles (see FIGS. 6 and 13). Thus, in general, synthetic HAP is characterized as having low crystallinity, high porosity, and high heterogeneity.

All implantable materials must be biocompatible, meaning they should not elicit a local or systemic immune response. Further, it is desirable that the HAP used in implants be bioresorbable so that it can be replaced gradually with regenerated bone. Currently available synthesized HAP is typically highly stable, which significantly impedes the rate of bone regeneration when it is used as a hard tissue replacement material. Further, processing conditions such as high pH, high temperature, and ultra-sonication often render synthetic HAP with other properties that deviate from natural HAP, limiting the bioactivity of the product.

Many of the drawbacks of synthesized HAP can be avoided by using naturally produced HAP, which has superior biocompatibility, biodegradability, and bioactivity. Naturally produced HAP can be extracted from sources such as eggshells, coral, fish bone, chicken bone, and body fluids. In one common method, for example, HAP is prepared from eggshell in a phosphate solution at a high temperature. Unfortunately, while the HAP obtained from such sources has superior properties (i.e., crystallinity and homogeneity), the methods required to extract it are extremely laborious and time consuming.

One appealing alternative method involves synthesizing HAP in cell culture. It has long been held that mesenchymal stem cells must fully differentiate into osteogenic precursors to be competent for biomineralization. Thus, the current methods for culture-based, ex vivo synthesis of HAP involve inducing fully differentiated osteogenic cells with minimal essential medium (conditioned MEMα) containing a source of inorganic phosphates, ascorbic acid (Vitamin C), and fetal bovine serum (≥20%) [20,21]. Unfortunately, standard methods of in vitro differentiation require a long incubation period (3-4 weeks) and are hampered by unpredictable outcomes. Thus, there is a need in the art for improved, efficient methods of producing HAP.

### SUMMARY

The present invention provides methods of making hydroxyapatite (HAP). In one aspect, the methods involve (a) providing cells expressing an alkaline phosphatase or genetically engineering cells to express an alkaline phosphatase, and (b) contacting the cells with calcium and an acyclic alkane phosphoester salt or inorganic phosphate salt such that the cells produce HAP. The contacting step is optionally in vitro or ex vivo.

In another aspect, the methods of making HAP involve contacting cells with calcium and an acyclic alkane phosphoester salt or inorganic phosphate salt such that the cells produce HAP. The cells may be cells that do not express alkaline phosphatase. Alkaline phosphatase may be added to the cells to improve uptake and transfer of the inorganic phosphate salt or in combination with the acyclic alkane phosphoester salt.

The methods may further comprise either harvesting the HAP produced by the cells or incubating the cells with an object and allowing the HAP to collect on and/or coat the object.

Additionally, the present invention provides methods of collecting HAP from cells. These methods involve (a) fixing the cells with an aldehyde and collecting the fixed cells, (b) washing the fixed cells with a basic solution and collecting the pellet, and (c) extracting the pellet with acetone or chloroform.

In another aspect, the present invention provides HAP produced by the methods disclosed herein. Preferably, the HAP has a crystalline structure and comprises crystallite particles that are between 0.1 nm and 40 nm in size.

In another aspect, the present invention provides methods of using the HAP produced by the methods disclosed herein. These methods involve contacting an object, such as a collagen, pharmaceutical agent, medical device, scaffold or implant, with the HAP.

Additionally, the present invention provides methods of measuring organic phosphates in a sample from a subject. The methods involve (a) obtaining a sample from the subject, (b) preparing a supernatant from the sample, (c) heat inactivating a portion of the supernatant of step b, (d) incubating the supernatant of step b and the product of step c with alkaline phosphatase for at least 2 hours, and (e) performing a phosphorus detection assay and comparing the treated supernatant of step b with the heat inactivated supernatant of step c, wherein the difference equals the quantity of organic phosphates in the sample.

The present invention also provides methods of measuring glycerophosphates in a sample from a subject. The methods involve (a) obtaining a sample from the subject, (b) preparing a supernatant from the sample, (c) incubating the supernatant with a detectable substrate and a glycerophosphate oxidase, and (d) measuring the detectable substrate of the reaction of step c.

In a final aspect, the present invention provides kits for measuring glycerophosphates in a sample from a subject. The kits comprise an oxidase, a glycerophoshate standard, and a detectable substrate capable of detecting hydrogen peroxide.

### BRIEF DESCRIPTION OF THE DRAWINGS

FIG. 1 demonstrates that calcium ions (Ca²⁺), a phosphoester salt, and alkaline phosphatase (ALP) are essential for promoting biomineralization in MG-63 and Saos-2 cell lines and human blood mononuclear cells (MNCs). (A) Alizarin Red S assays (ARS) show mineralization within 7 days, visualized as the intensity of red staining, of Saos-2 cells in MEMα/10% FBS supplemented only with βGP or the combination of βGP/ascorbic acid (Vit. C)/dexamethasone (Dex); MG-63 cell line was inactive under similar conditions. No mineralization was observed in either cell line cultured with MEMα/10% FBS supplemented with Vit. C or Dex. In MEMα/10% FBS supplemented with βGP and ALPL, calf intestinal ALP (CIP), or shrimp ALP (SAP), MG-63 and Saos-2 cell lines were mineralized. (B) Similar results were iterated when αGP was used instead of βGP. (C) Compared to βGP and phosphoenolpyruvate monosodium (PEP), αGP was the most efficient phosphoester salt elicited biomineralization. Pamidronate (Pamidn) and glycerophosphoric acid (NSC9231) did not elicit the reaction. (D) Human MNCs also have an innate ability of mineralization in 7 days without the induction of cellular differentiation (top row indicates initial cell counts per well in a 6-well plate). (E-G) Titration assays indicated that biomineralization depended on the doses of αGP, CIP, and Ca²⁺. (H) In a 48-well plate, human blood MNCs were seeded (10⁵/well) and exposed to MEMα/10% FBS, the medium supplemented with αGP, with αGP and CIP, or with CIP for 7 days (the media were changed on day 4). On day 7, ARS indicates that biomineralization occurred in MNCs exposed to MEMα/10% FBS supplemented with αGP and CIP in the uncoated wells (top row) and the wells coated with Collagen Type I, rat tail (middle row). Biomineralization did not occur if αGP or CIP was missing. None of the cell-less wells coated with Collagen Type I was positive for HAP by ARS (bottom row). Each of the tests was in duplicates.

FIG. 2 shows biomineralization of human cells. Saos-2 cells were grown at low confluency on a glass slide immersed in MEMα/10% FBS with αGP (2 mM) for 24 h (24HR) and 96 h (96HR), respectively. The slides were washed, fixed, and stained with Alizarin Red S. The cell morphology was illustrated using optical phase-contrast (with pseudo-green background), and HAP minerals were in red under a ZEISS inverted microscope equipped with an Infinity 3 digital camera and imaging software. Cells were magnified 400×.

FIG. 3 shows electron micrographs of biomineralization. K562 cells were cultured for 72 h in MEMα containing 10% FBS, αGP (2 mM), and CIP (1 U/mL). (A1-A2) Formation of caveolae (yellow arrows) initiated endocytosis of mineral matrix at the cytoplasmic membrane. (B-C) Caveolar endocytosis (red arrows) transported the mineral matrixes to the endosomes (e), where calcium phosphate agglomerates were synthesized (blue arrows; M=mitochondrion). (D) Endosomes (e) budded from the cell membrane (green arrows) to release the mineral agglomerates (blue arrows) into the extracellular space. Bars indicate various scales within the images.

FIG. 4 shows electron micrographs depicting two types of amorphous calcium phosphate (ACP) precursors. (A) Large spherical particles (50-100 nm) with remarkable electron-dense areas (inset, yellow arrows) are typical of chemically synthetized nanoparticles (scale bar=50 nm). (B-D) Biologically synthetized agglomerates of much smaller granules composed of hydroxyapatite (HAP) crystallites (5-10 nm; blue arrow) produced by human cells grown for 7 days in MEMα/10% FBS with αGP (2 mM) and CIP (1 U/mL). Scale bars: (B)=100 nm; (C)=20 nm; (D)=2 nm. (E) Boundaries (green arrows) formed between HAP grains (scale bar=5 nm).

FIG. 5 shows the development of hydroxyapatite (HAP). (A) Amorphous calcium phosphate (ACP) coiling (yellow arrows) was visible at a remarkable precrystalline stage that formed polycrystalline masses (PC, in yellow circle). (B) Primary crystallization events occurred at the center of the coiling polycrystalline mass (yellow arrows) and resulted in 5-10 nm focus with the crystallographic texture of HAP (hollow arrows; scale bar=5 nm). (C) Primary crystallization triggered a chain reaction that expanded HAP to crystallite grains (white arrows and numbers discrete HAP crystallites; scale bar=2 nm).

FIG. 6 shows an X-ray diffraction (XRD) analysis of the composition of nanoparticles generated by cell biomineralization versus industrial production. Panel A: The mineral extracted from human blood mononuclear cells (HAP Biomineralization) cultured in MEMα/10% FBS supplemented with αGP (2 mM) and CIP (1 U/mL) for 7 days was identified as homogeneous hydroxyapatite (HAP) (Ca₁₀(PO₄)₆(OH)₂, blue). The industrial product of recovered bony material was identified as heterogeneous calcium-phosphate hydrate (Ca₃(PO₄)₂·xH₂O) (HA Commercial; black). Panel B: XRD analysis for materials recovered from animal bones by calcination and from Sol-gel chemical synthesis. Panel C: XRD analysis for nanoparticles recovered from human adherent cells (red), human mononuclear cells (MNC; blue), and suspension cells (green).

FIG. 7 shows gene expression profiling and western blot detection of human alkaline phosphatase (ALP) in cell lines. (A) TaqMan quantitative RT-PCR of human ALPs (ALPG, ALPI, ALPL, and ALPP) in hFOB1.19, MG63, and Saos-2 cell lines. Error bars indicate the standard deviations of three individual RT-PCR assays in triplicate. (B) Western blot of ALPL and GAPDH in 13 human cell lines (20 μg of total protein from cell lysate per lane).

FIG. 8 demonstrates that biomineralization of C2C12 and MCF-7 cells was dependent on αGP (2 mM) and CIP (1 U/ml) supplemented MEMα/10% FBS with ascorbic acid (Vit. C) or without ascorbic acid (ØVit. C). The cells were cultured for 7 days (the media were changed on day 4) and then stained with Alizarin Red S.

FIG. 9 shows biomineralization of human and mouse cells without induction of cellular differentiation. Alizarin Red S staining revealed mineralization in (A) human cell lines (n=28) and (B) mouse cell lines (n=2) after 7 days of culture in MEMα/10% FBS supplemented with αGP (2 mM) and CIP (1 U/ml), the media were changed on day 4. The reaction did not occur if any one of the three elements was missing.

FIG. 10 shows an alignment of amino acid sequences of human tissue-nonspecific ALP (ALPL; SEQ ID NO:1), calf intestinal ALP (CIP; SEQ ID NO:2), and shrimp hepatopancreas ALP (SAP; SEQ ID NO:3). ALPs from three distant species across Kingdom Animalia demonstrate substantial differences; 48 residues are conserved (*).

FIG. 11 demonstrates that titanium plates may be coated with HAP using the cell culture-based methods disclosed herein. (A) Calcium phosphate biomineralization in human mononuclear cells (MNC) from the peripheral blood of four adult donors (Donor 01-04) grown in MEMα/10% FBS with added αGP (2 mM) and CIP (1 U/ml) for 7 days and stained with Alizarin Red S. (B) Titanium surfaces incubated with the MNC cells described above, and an untreated surface (left) for comparison. After 14 days, the titanium surfaces incubated with the MNC cells were coated in secreted HAP.

FIG. 12 illustrates a quantitative assay that the inventors developed for measuring the concentration of glycerophosphate in samples obtained from a subject. (A) αGP standards with concentrations ranging from 0.15625 mM to 10 mM subjected to a photometric assay catalyzed by horseradish peroxidase (HRP). (B) Optical density readings of these reactions taken at a wavelength of 500 nm (OD500). (C) Standard curves produced from the measured data.

FIG. 13 shows the results of nanoparticle tracking analysis (NTA) performed on nanoparticles from human somatic cells that have undergone biomineralization in MEMα containing FBS, αGFP, and ALP (HAP_BioM) and three commercial sources (HA_C001, HA_C002, and HA_C003). The x-axis indicates size in nanometers.

FIG. 14 demonstrates that cell proliferation was not adversely affected by biomineralization. Four human myeloma cell lines were exposed to MEMα/10% FBS containing αGP, αGP+CIP (red), or CIP for 7 days. HAP minerals were stained with Alizarin Red. Cell proliferation was measured by MTT assay on days 2, 4, and 7.

FIG. 15 demonstrates that in vitro biomineralization can be used to coat a poly ε-caprolactone (PCL) scaffold (MilliporeSigma Co). Biomineralization was performed in peripheral blood mononuclear cells (MNCs) grown in MEMα supplemented with 10% FBS, 2 mM of αGP, and 1 U/ml of CIP. (A) Uncoated scaffold at day 0. The purple arrows mark the original width of PCL wires. (B) The scaffold was coated and partially filled with the solid materials produced by MNCs on day 20. (C) On day 20, the coated scaffold, was stained with Alizarin Red S to confirm that the filling material is HAP. (D) Comparison of an uncoated PCL scaffold (left) to a HAP-coated PCL scaffold (right).

### DETAILED DESCRIPTION

Biomineralization is of primary importance in the formation of bones and teeth. In this process, hydroxyapatite (HAP) is deposited in the extracellular space of the collagen by specialized bone cells called osteoblasts. HAP, a naturally occurring crystalline form of calcium phosphate, is the main component of bones and teeth, forming 70% of human bone by weight and 70-80% of the mass of dentin and enamel in teeth. While this mineral has the formula Ca₅(PO₄)₃(OH), it is usually written “Ca₁₀(PO₄)₆(OH)₂” to denote that the crystal unit cell comprises two entities.

In the present application, the inventors demonstrate that biomineralization is a natural ability that is shared by all somatic cells. Regardless of differentiation status, eukaryotic cells respond to the simultaneous presence of calcium, glycerophosphate, and alkaline phosphatase by producing amorphous calcium phosphate precursors that transform into crystalline HAP.

### Methods of HAP Production

The present invention provides methods of making HAP. These methods represent a substantial improvement over traditional protocols. As is detailed in the Introduction, chemically synthesized HAP comes with several drawbacks including reduced bioactivity, and current methods for preparing HAP from natural sources are laborious and time consuming, requiring incubation times of several months. With the methods of the present invention, HAP can be efficiently produced from cultured cells within one week.

In one aspect, the methods involve contacting cells with calcium and an acyclic alkane phosphoester salt or inorganic phosphate salt such that the cells produce HAP. The cells used with these methods may be cells do not express alkaline phosphatase, such as non-osteoblast cells. As used herein, a “non-osteoblast cell” refers to cells that are not derived from an osteoprogenitor cell. These cells do not spontaneously produce HAP under normal physiological conditions, and must be provided with sufficient levels of calcium and a phosphate source to do so. In some embodiments, the methods further comprise contacting the cells with an alkaline phosphatase. However, the inventors have discovered that alkaline phosphatase is not required for cells to produce HAP if they are contacted with a high concentration of inorganic phosphate salt (e.g., more than 1 mM, suitably at least 2 mM and up to 1M). Alkaline phosphatase may be added to the cells to improve uptake and transfer of the inorganic phosphate salt and then lower concentrations of the inorganic phosphate salt are required to produce HAP. Alternatively, if cells make alkaline phosphatase or if the alkaline phosphatase is provided in trans, then acyclic alkane phosphoester salts in combination with calcium are sufficient to allow the cells to produce HAP.

In another aspect, the methods utilize cells that express an alkaline phosphatase. The cells are contacted with calcium and an acyclic alkane phosphoester salt or inorganic phosphate salt, such that the cells produce HAP. This contacting step is optionally performed in vitro or ex vivo. In some embodiments, the cells used with these methods naturally express an alkaline phosphatase. In other embodiments, the cells are genetically engineered to express an alkaline phosphatase.

As used herein, “genetically engineering” refers to the process of artificially introducing a genetic modification. Genetic engineering can be performed at the DNA, RNA, or epigenetic level. Genetic modifications include: (i) deletion of an endogenous gene; (ii) introduction of a recombinant nucleic acid encoding a wild-type or mutant form of an endogenous or exogenous protein; (iii) introduction of an RNA molecule (e.g., small-interfering RNA (siRNA), short hairpin RNA (shRNA), anti-sense RNA, and micro RNA (miRNA)) that interferes with the functional expression of a protein; or (iv) altering the promoter or enhancer elements (i.e., regulatory elements) of one or more endogenous genes. It is understood that item (ii) includes replacement of an endogenous gene (e.g., by homologous recombination) with a gene encoding an altered or entirely different protein, and that item (iv) includes modification or manipulation of the regulatory regions of a target gene or of any region that is contiguous with a target gene (e.g., up to 5 KB on either side of the target sequence). Genetic engineering also includes altering an endogenous gene to produce a protein having additions (e.g., a heterologous sequence), deletions, or substitutions (e.g., mutations such as point mutations; conservative or non-conservative mutations). Mutations can be introduced specifically (e.g., by site-directed mutagenesis or homologous recombination) or can be introduced randomly (e.g., chemically mutagenized). Thus, genetic modifications may modulate a gene in several ways, such as increased-expression, increased function, reduced-expression, reduced function, or gene knockout.

In some embodiments of the present methods, cells are genetically engineered to express one or more alkaline phosphatase. In some embodiments the cells used with the present invention are genetically engineered to express an exogenous alkaline phosphatase gene by introducing a recombinant nucleic acid encoding the exogenous gene into the cell. In other embodiments, the cells may be engineered to express an endogenous alkaline phosphatase gene, for example, by altering regulatory elements of the gene, introducing an extra copy of the gene, or introducing another gene or nucleic acid sequence that regulates the expression of the target gene (e.g., a transcription factor or exogenous promoter).

Genetic engineering is performed using several methods that are known in the art. Using these methods, new genetic material may be introduced into the cell directly (i.e., via injection, encapsulation, or electroporation) or delivered via another cell, liposome or a virus that is then fused with the cell. Genetic engineering methods may involve use of engineered nucleases (e.g., meganucleases, zinc finger nucleases (ZFN), transcription activator-like effector nucleases (TALENs), and the Cas9-guideRNA system (adapted from CRISPR). In some embodiments, genetic engineering involves altering the nuclear genome of the cell. When new genetic material is introduced to the nuclear genome, it can be inserted randomly or targeted to a specific location (e.g., via homologous recombination). In other embodiments, the engineered cell may harbor a vector comprising a target gene that is expressed independently of the nuclear genome.

Genetic engineering can be performed using conventional techniques of molecular biology, microbiology, and recombinant DNA, which are within the skill of the art. Such techniques are explained fully in the literature. See, for example, Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

As used herein, contacting includes contacting cells directly or indirectly in vivo, in vitro, or ex vivo. Contacting encompasses administration to a cell, tissue, mammal, patient, or human. Further, contacting a cell includes adding an agent to a cell culture system. Other suitable methods may include introducing or administering an agent to a cell, tissue, mammal, or patient using appropriate procedures and routes of administration as defined above.

### Cell Culture

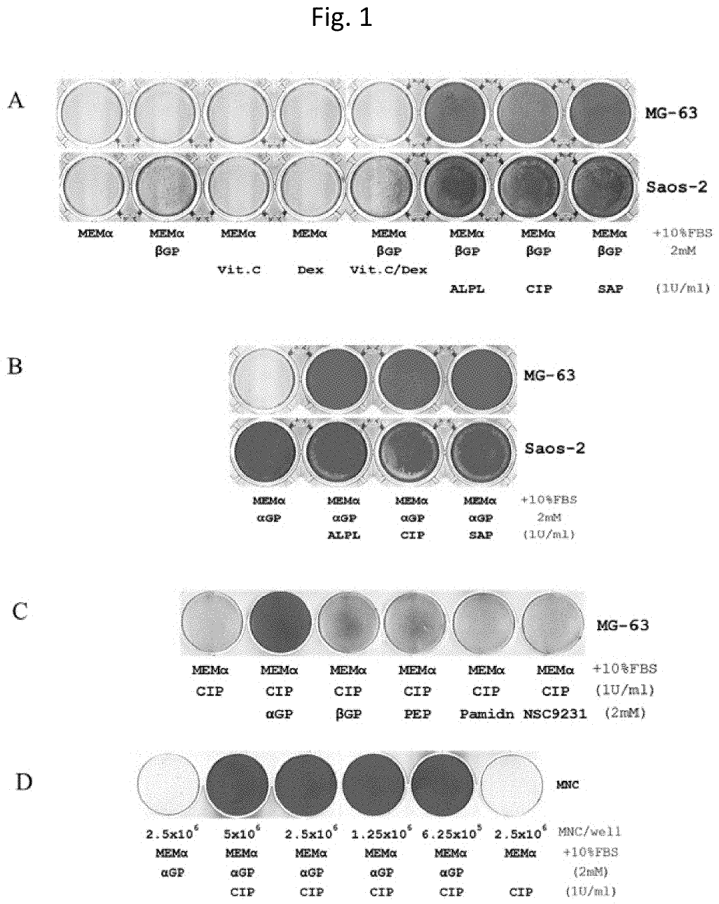

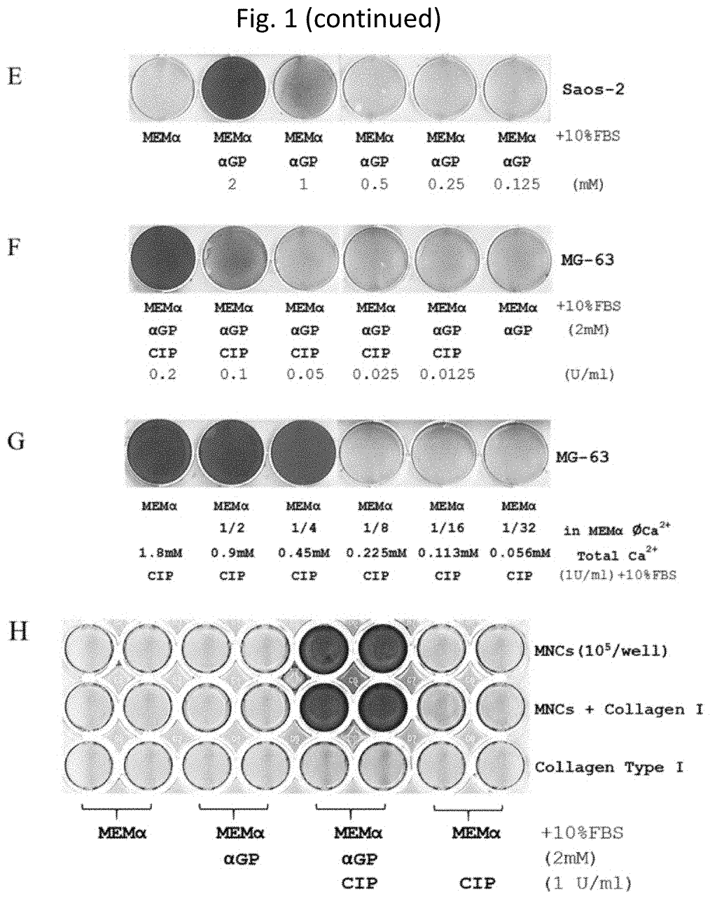

Exemplary cell types for use with the present invention include, for example, bone cells, stem cells, blood cells (e.g., mononuclear cells), muscle cells, fat cells, skin cells, nerve cells, endothelial cells, and pancreatic cells. In some embodiments, the cells are mononuclear cells derived from whole blood. As used herein, the term “mononuclear cells” or “MNCs” refers to blood cells with a single, round nucleus, such as lymphocytes (e.g., T cells, B cells, NK cells) and monocytes. Mononuclear cells can be isolated from peripheral blood or bone marrow of a subject. Any of these individual cell types may be used in the methods or any combination of these cells may be used in the methods provided herein. In addition to human cells, cells from other species (e.g., domestic fowl, bovine, goats, and sheep) can also be utilized for large-scale HAP manufacturing. In the Examples, the inventors demonstrate that several human and murine cell lines possess the ability to perform biomineralization in vitro, including cells derived from osteoblast, bone marrow stroma, embryo, muscle, myoblast, fibroblast, neuron, as well as malignant cell lines derived from breast, colon, prostate, cervix, leukemia/lymphoma, and myeloma plasma cells. All of these cells produced calcium mineral deposits, which were detected by Alizarin Red S staining (see, e.g., FIG. 1). Thus, the inventors hypothesize that the ability to perform biomineralization is innate to all eukaryotic cells, regardless of cell type, origin, and maturity.

The cell lines assayed in the Examples include: MG63, Saos-2, hFOB 1.19, HS-5, HEK-293, RD, HCN-2, MDA-MB-231, MCF-7, SW480, SW620, Colo205, Colo32DM, Du145, LNCap FGC, PC-3, HeLA, H929, ARK, ARP1, CAG, JJN3, OPM2, RPMI8226, U266, HL-60, K-562, THP1, U937, C2C12, and NIH-3T3. Many of the assayed cell lines comprise transformed somatic cells. As used herein, the term “somatic cell” refers to any cell of a living organism other than the reproductive cells. As used herein, the term “transformed cell” or “cancerous cell” refers to any immortalized or cancer-derived cell line. Further, while most of the assayed cell lines are of human origin, two of these lines (C2C12 and NIH-3T3) are of mouse origin. Thus, while the cells utilized in the present invention may be derived from any eukaryote, mammalian cells are utilized in preferred embodiments.

The methods of the present invention may be performed in vitro or ex vivo. The terms “in vitro” and “ex vivo” both refer to a process performed outside a living organism. As used herein, the term ex vivo refers to methods that use cells collected from a subject, which cells may be returned after completion of the method. The term in vitro refers to methods using cell lines or harvested, isolated cells in tissue culture only. Many cell lines are available to those of skill in the art for use in vitro. In preferred embodiments, the methods involve inducing biomineralization in cells grown in culture. The present invention encompasses the use of any appropriate media, supplements, incubators, cell culture vessels, and substrates that meet the cells' basic requirements for nutrients, temperature (i.e., about 37° C.), carbon dioxide (i.e., about 5%), and atmospheric oxygen.

Exemplary nutrient sources for the cultured cells used in the practice of the present invention may include an energy source such as glucose, fructose or galactose; both essential and nonessential amino acids; both water-soluble (B group, biotin, folic acid, nicotinamide, panthothenic acid, pyroxidine, riboflavin and thiamine) and fat-soluble (A, D, E, K, and ubiquinone) vitamins; major inorganic ions such as bicarbonate, calcium, chloride, magnesium, phosphate, potassium, lithium, and sodium; trace elements such as As, Co, Cr, Cu, F, Fe, Mn, Mo, Ni, Se, Si, Sn, V and Zn; lipids; buffers, e.g., like CO₂/HCO₃ and HEPES; gases (oxygen and carbon dioxide); and nucleic acid precursors like adenine, cytidine, hypoxanthine, and thymidine. There are many commercially available media which are expected to be useful in the practice of the present invention, including, for example, but not limited to Dulbecco's Modified Eagle's Medium (DMEM), Minimum Essential Medium (MEM), M199, RPMI 1640, and Iscove's Modified Dulbecco's Medium (EDMEM, Gibco Labs). However, in preferred embodiments, the cells are grown in a standard osteogenic medium, such as conditioned Minimum Essential Medium a (MEMα). Those of skill in the art can identify the appropriate media for the cell type chosen.

The medium may be supplemented with any serum, serum replacement, albumin, amino acids, proteins, lipids, hormones, vitamins, nucleic acids, buffers, reducing agents, salts, or growth factors that are deemed desirable given, e.g., the particular medium in use and cells being cultured. For example, MEMα is commonly supplemented with heat-inactivated 10% Fetal Bovine Serum (FBS) or 10% horse serum. Exemplary supplements include insulin or an insulin-like growth factor, epidermal growth factor (EGF), transferrin or ferrous ion, triiodothyronine or thyroxin, ethanolamine and/or o-phosphoryl-ethanolamine, hydrocortisone, strontium, progesterone, selenium, phospholipid precursors, enzyme cofactors, inorganic salts, fatty acids, cholesterol, pyruvic acid, β-mercaptoethanol, and cAMP elevating agents.

In particular embodiments, the medium is a “conditioned medium”. Conditioned medium is prepared by culturing a first population of cells in the medium before it is collected and used to grow a second population of cells. Thus, conditioned medium contains metabolites, growth factors, and extracellular matrix proteins secreted by the first population of cultured cells.

The methods of the present invention can be performed in any suitable culture vessel or bioreactor. As used herein, the term “bioreactor” refers to any manufactured device or system that supports a biologically active environment. Exemplary vessels include, for example, Tissue culture flasks, multi-well plates, spinner flasks, culture tubes, roller bottles, and petri dishes. Many commercially available cell culture vessels include features that are beneficial for growing cells, such gas permeable materials.

Those of skill in the art are familiar with standard cell culture methods and understand that different cell lines have unique requirements. For instance, some cells benefit from growing with or on “feeder cells” or on various substrates, such as collagen, fibronectin, laminin, or heparan sulfate proteoglycan. Detailed cell culture methods can be found in literature references, such as Culture of Animal Cells: A Manual of Basic Technique (RI Freshney ed., Wiley & Sons) and General Techniques of Cell Culture (MA Harrison and IF Rae, Cambridge Univ Press), and at the websites of commercial suppliers, such as Thermo Fisher Scientific and Sigma-Aldrich.

### Induction of Biomineralization

To induce biomineralization using the methods of the present invention, cells are grown in the presence of at least two factors: calcium and a suitable phosphate source (i.e., acyclic alkane phosphoester salt or inorganic phosphate salt). Optionally, the cells are also grown in the presence of an alkaline phosphatase isozyme. The alkaline phosphatase may be added to the culture medium or may be expressed by the cells (i.e., cells that either naturally express alkaline phosphatase or cells that were engineered to produce alkaline phosphatase).

Calcium may be provided as a component of the culture medium (e.g., the MEMα used in the Examples contains calcium), or as a supplement added to the culture medium. Calcium is frequently added to cell culture media as freely soluble calcium chloride. Other examples of calcium sources include calcium oxide, calcium carbonate, calcium hydroxide, calcium hydroxide-calcium carbonate double salts, or a basic calcium phosphate or mixtures thereof.

In the Examples, the inventors demonstrate that the requirement for calcium in biomineralization is concentration dependent. Thus, in preferred embodiments, the cells are contacted with 0.02-2 mM calcium. Suitably, the concentration of calcium is at least 0.2 mM, at least 0.3 mM, at least 0.4 mM, at least 0.45 mM, at least 0.5 mM.

As used herein, the term “acyclic alkane phosphoester salt” refers to any phosphoester salt with an acyclic alkane backbone. Exemplary acyclic alkane phosphoester salts include, without limitation, disodium β-glycerophosphate, disodium α-glycerophosphate, phosphoenolpyruvate sodium, and disodium or dilithium dihydroxyacetone phosphate (DHAP).

As used herein, the term “inorganic phosphate salt” refers to a salt of phosphoric acid with metal ions. Notably, inorganic phosphate is a major component of hydroxyapatite in bone. Any inorganic phosphate may be used with the methods disclosed herein. For instance, a sodium phosphate salt may be utilized.

Any acyclic alkane phosphoester salt, inorganic phosphate salt, or combinations thereof may be utilized with the present invention. However, in preferred embodiments, the acyclic alkane phosphoester salt α-glycerophosphate (αGP) is utilized, which was shown to induce biomineralization more efficiently than all other salts tested in the Examples. Glycerophosphate refers to an anion of a phosphoric ester of glycerol, in which a carbon atom of glycerol bonds to an oxygen atom in the phosphate group of phosphoric acid. In the cell, glycerophosphate and DHAP serve as a major link between carbohydrate metabolism and lipid metabolism, which both contribute to energy production by the electron transport chain within mitochondria. There are two structural isomers of glycerophosphate, referred to as the α and β isomers. In the α isomer, the phosphate radical is attached to the first or third carbon on the glycerin chain, and in the β form, the phosphate is attached to the second (middle) carbon. Glycerophosphate can be a chiral molecule, i.e., it can exist in two forms that are nonsuperimposable mirror images. It is intended that the present invention includes within its scope both isomeric forms of a glycerophosphate and/or their racemates.

Exemplary glycerophosphate salts include calcium glycerophosphate, magnesium glycerophosphate, ammonium glycerophosphate, zinc glycerophosphate, manganese glycerophosphate, lithium glycerophosphate, cupric glycerophosphate, ferric glycerophosphate, quinine glycerophosphate, sodium glycerophosphate, potassium glycerophosphate, barium glycerophosphate, and strontium glycerophosphate. Glycerophosphate may also be obtained as an injectable solution (Glycophos™, Fresenius Kabi, Lake Zurich, Ill.).

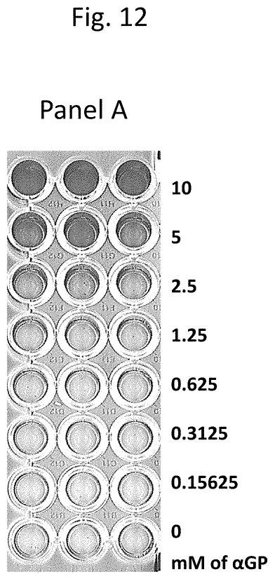

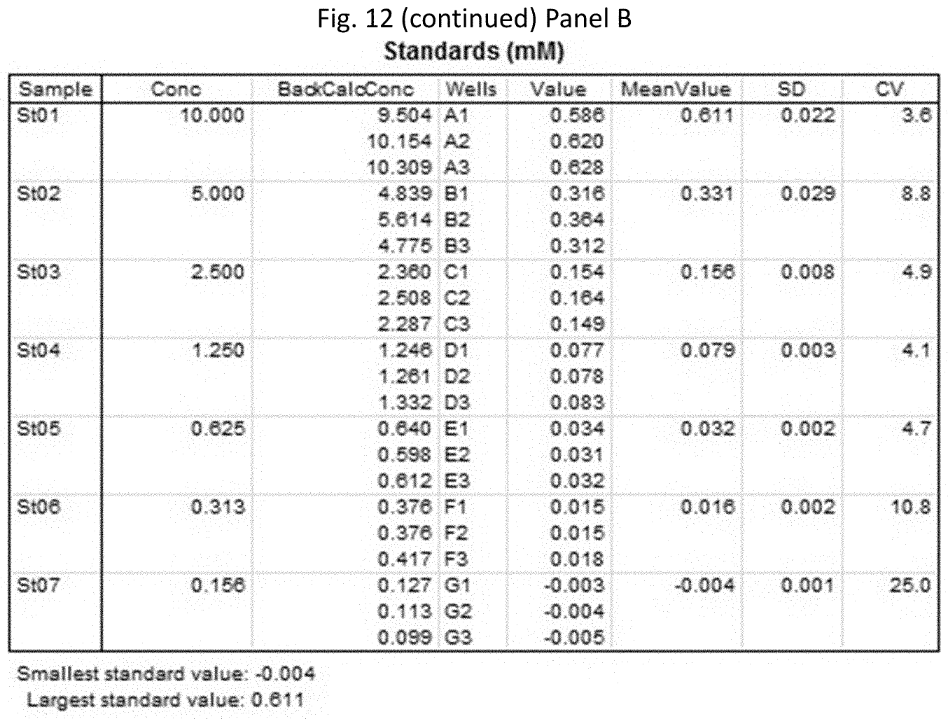

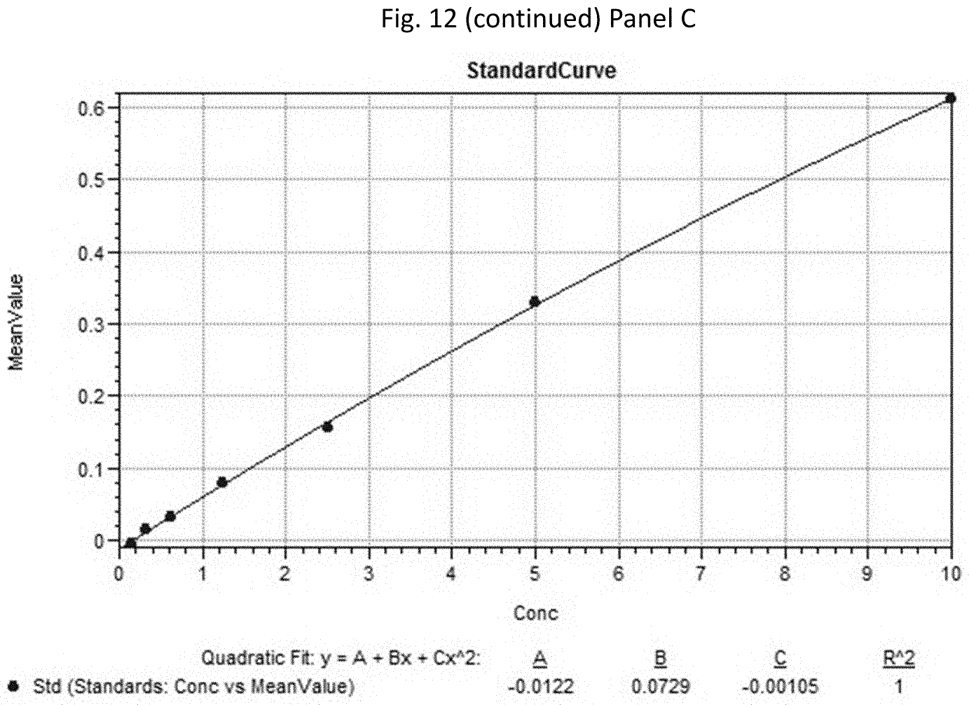

In the Examples, the inventors demonstrate that the requirement for a phosphate source in biomineralization is concentration-dependent. Thus, in preferred embodiments, the cells are contacted with 0.5-5 mM acyclic alkane phosphoester salt. Suitably, the concentration of phosphoester salt is at least 2 mM. However, when inorganic phosphate is utilized as a phosphate source, a greater concentration will likely be required. Suitably, the cells are contacted with 0.001-1M of inorganic phosphate. The inventors have also developed a quantitative technique for measuring the concentration of glycerophosphate in samples obtained from a subject, such as urine or serum. In the described assay, the oxidation of glycerophosphate releases hydrogen peroxide (H₂O₂), which can be measured using horseradish peroxidase (HRP) to catalyze photometric assays and convert colorimetric or fluorescent substrates for detection and quantification (see FIG. 12).

Alkaline phosphatase (ALP) is a hydrolase enzyme that removes phosphate groups from many types of molecules, including nucleotides, proteins, and alkaloids. These enzymes not only catalyze the hydrolysis of monoesters of phosphoric acid, but also catalyze a transphosphorylation reaction in the presence of high concentrations of phosphate acceptors. As is indicated by their name, alkaline phosphatases are most effective in an alkaline environment. While the mechanism by which this enzyme functions in biomineralization is not completely understood, ALP may act both to increase the local concentration of inorganic phosphate (which promotes mineralization) and to decrease the concentration of extracellular pyrophosphate (which inhibits mineral formation). However, work by inventors suggests that the role of ALP in biomineralization is independent of phosphatase activity. Thus, the use of an ALP lacking phosphatase activity is also contemplated herein. ALP is attached to the outside of the membrane of cells and of matrix vesicles via a glycophosphatidylinositol anchor, and is found within membrane microdomains known as lipid rafts. Thus, a possible role for ALP in biomineralization is that it promotes the internalization of calcium and glycerophosphate into cells. The inventors believe transport function is likely how ALP contributes to this process. Thus, mutant ALP proteins lacking phosphatase activity, but maintaining the ability to transport calcium and glycerophosphate may be used in the methods described herein.

In the Examples, the inventors demonstrate using titration experiments that the requirement for ALP in biomineralization is concentration-dependent in the presence of relatively low amounts of a suitable phosphate source, though very little alkaline phosphatase is required. Their results suggest that the presence of endogenous ALP (e.g., in Saos-2 cells) or the limited amount of ALP provided as a component of fetal bovine serum (FBS) is sufficient to induce biomineralization in eukaryotic cells during a 3-4 week incubation [20-21]. Further, the inventors recently discovered that alkaline phosphatase is not required for cells to produce HAP if they are contacted with a high concentration of inorganic phosphates (e.g., more than 2 mM and up to 1M). Thus, depending on the concentration of phosphates used, very little to no ALP is required to practice the methods of the present invention.

In certain embodiments, the ALP is added to the medium. Preferably, in these embodiments, the cells are contacted with 0.05-0.5 U/ml of ALP. In other embodiments, ALP expression is induced in the cultured cells through genetic engineering, such that the addition of ALP is not required. Here, the genetic engineering may involve upregulation of an endogenous gene or introduction of a transgene (described in more detail above).

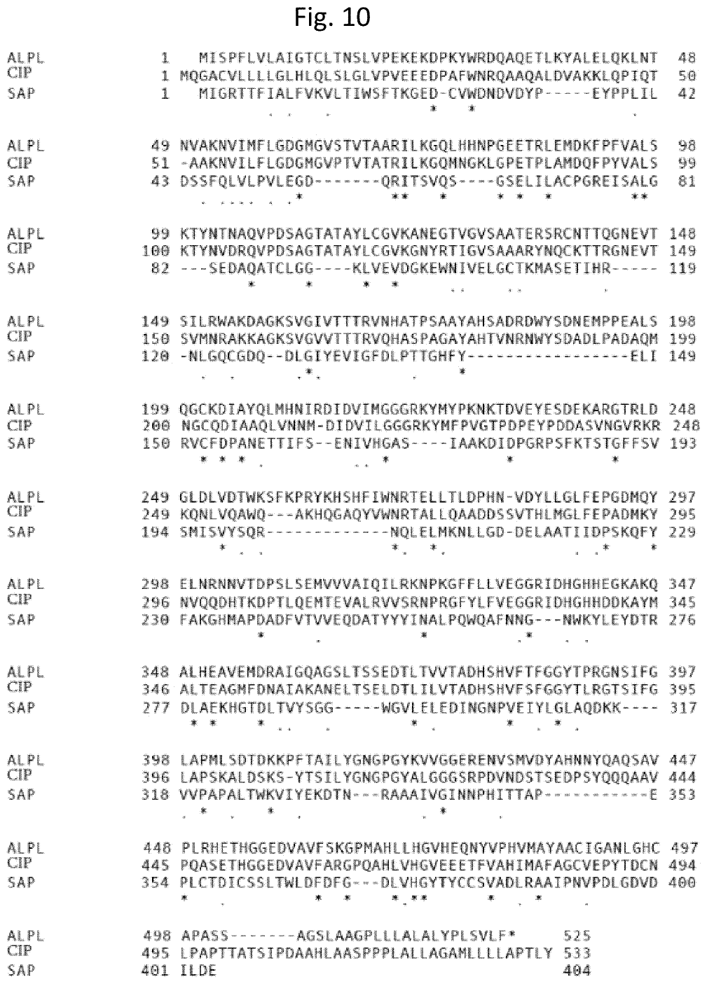

The human genome encodes four distinct ALP enzymes: intestinal alkaline phosphatase (ALPI), germ cell alkaline phosphatase (ALPG), placental alkaline phosphatase (ALPP), and tissue-nonspecific alkaline phosphatase (ALPL). ALPI, ALPG, and ALPP are closely related (86% amino acid sequence identity) and are clustered within the genome at chromosome 2q37.1. In contrast, ALPL is located at chromosome 1p36 and has less than 50% amino acid sequence identity with the other three ALPs (FIG. 10). While ALPI, ALPG, and ALPP are generally inactive, ALPL is expressed in bone, liver, kidney, brain, skin, and vascular endothelial cells [26], which are frequent sites of cancer metastasis and ectopic calcification. ALPL differs from the other human ALPs in that it contains both a domain that specifically binds calcium ions and a C-terminal segment for anchoring to the phosphatidylinositol (GPI)-glycan moieties found on cytoplasmic membranes [27-29]. At sites of bone formation, ALPL supplies inorganic phosphate (Pi) for mineralization by cleaving pyrophosphate (PPi) to inorganic phosphorus. As a result, loss-of-function mutations in ALPL cause hypophosphatasia (HPP), an inborn bone formation defect found in both children and adults [30, 31].

Alkaline phosphatase may be purified from a variety of bacterial, fungal, alga, invertebrate and vertebrate species. Notably, alkaline phosphatase from calf intestine and shrimp are commercially available and widely used in molecular biology and other applications. ALPL was previously considered to be the specific phosphatase required for bone formation. However, in the Examples, the inventors made the surprising discovery that biomineralization can be elicited in somatic cells not only by human ALPL but also by ALPs from other species, including cows, shrimp, and sheep. Thus, the present invention encompasses the use of an alkaline phosphate from any species, including but not limited to, bacterial alkaline phosphatase (BAP), shrimp alkaline phosphatase (SAP), calf intestine alkaline phosphatase (CIP), bovine intestinal alkaline phosphates (bIAP), and placental alkaline phosphatase (PLAP) and its C-terminally truncated counterpart: secreted alkaline phosphatase (SEAP). In some embodiments, the ALP used with the present invention is a recombinant protein. Recombinant ALP proteins are commercially available. For example, asfotase alfa (brand name STRENSIQ™; Alexion Pharmaceuticals) is a commercially available, recombinant, tissue nonspecific alkaline phosphatase that is used as a medication for the treatment of perinatal, infantile, and juvenile-onset hypophosphatasia. In preferred embodiments, the ALP is used with the present invention is alkaline phosphatase (ALPL), calf intestinal alkaline phosphatase (CIP), shrimp hepatopancreas alkaline phosphatase (SAP), or asfotase alfa.

One of skill in the art will appreciate that many modifications may be made to an enzyme such as ALP without significantly disrupting its function. Such modifications include insertions, deletions, or substitution of at least one amino acid as well as protein truncations. Additionally, functional moieties (e.g., fluorescent, epitope, or affinity tags) may be added to the enzyme to facilitate its detection or purification. Thus, the present invention encompasses the use of any ALP variant that provides the necessary function for biomineralization, namely the transport of calcium and glycerophosphatase.

The method provided herein also include harvesting the HAP produced by the cells in the methods. The HAP may be harvested after as little as 24 hours of incubation in the presence of calcium and a suitable phosphate source in the methods of the invention. The HAP may be harvested after 2, 3, 4, 5, 6, 7, 8, 9, 10, 11, 12, 13, 14, 15, 16, 17, 18, 19, 20, 25, or 30 days. In preferred embodiments, the HAP is harvested after at least 24 hours.

Instead of directly harvesting the HAP, materials or objects may be added to the cells to allow coating of the object or collection of HAP on the object. The optimal incubation time to allow an object to be coated with HAP is roughly 14-30 days, depending on cell type utilized. Objects to be coated may include, but are not limited to implantable medical devices, such as biomimetic bone or dental scaffolds composed of polylactic acid, polyglycolic acid, poly (lactic-co-glicolic acid), poly ε-caprolactone, polyethylene glycol, polybutylene terephthalate, polyethylene terephthalate, polyvinyl alcohol, polypropylene fumarate, poly aldehyde guluronate, poly acrylic acid, and polyurethane.

### Mineral Harvesting

The present invention also provides methods of collecting HAP from cells. The methods involve (a) fixing the cells with an aldehyde and collecting the fixed cells, (b) adding sodium hydroxide or another basic solution to the fixed cells and collecting the pellet, and (c) extracting the pellet with acetone or chloroform. The HAP may be harvested after any amount of time in culture, but as noted above at least 24 hours is generally required and generally the culture process should be less than a month, less than 2 weeks, or less than 1 week. In some embodiments, the HAP collected using these methods was made by the using the methods of making HAP disclosed herein.

As used herein “fixing” refers to the preservation of biological tissues from decay due to autolysis or putrefaction. Fixing may be accomplished using any aldehyde. However, in preferred embodiments, a 10% buffered formalin solution is utilized. In step (b), sodium hydroxide is added to remove nucleotides and cell debris. In some embodiments, the sodium hydroxide is provided as a 10% solution. The resulting pellet is then extracted with acetone. In some embodiments, the extraction is performed at least two times. After the pellet has been extracted in step (c), it may be washed one or more times with ethanol. Additional steps (e.g., drying, suspension in a suitable carrier) may be included in these methods to prepare the HAP for a specific downstream application.

### Compounds (Hydroxyapatite)

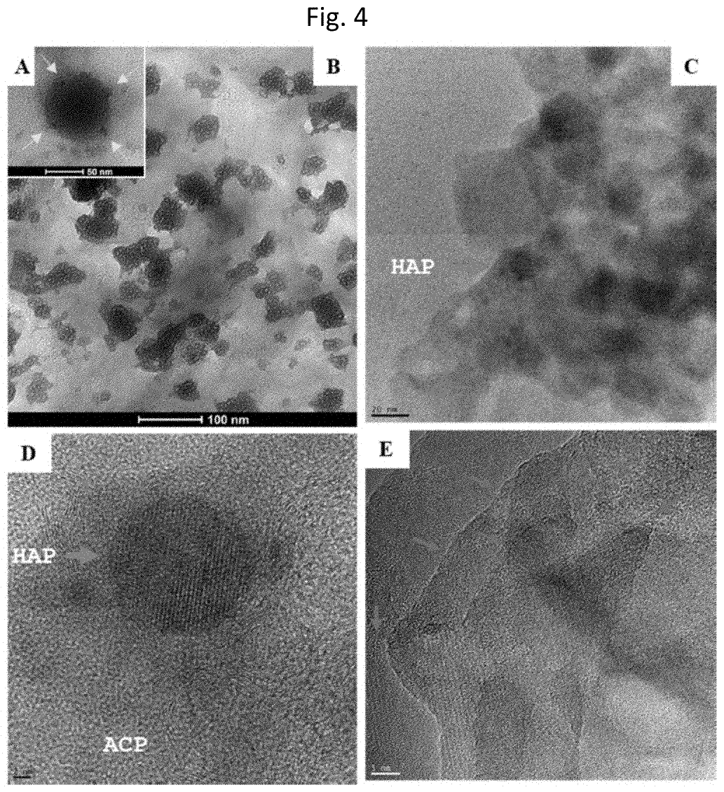

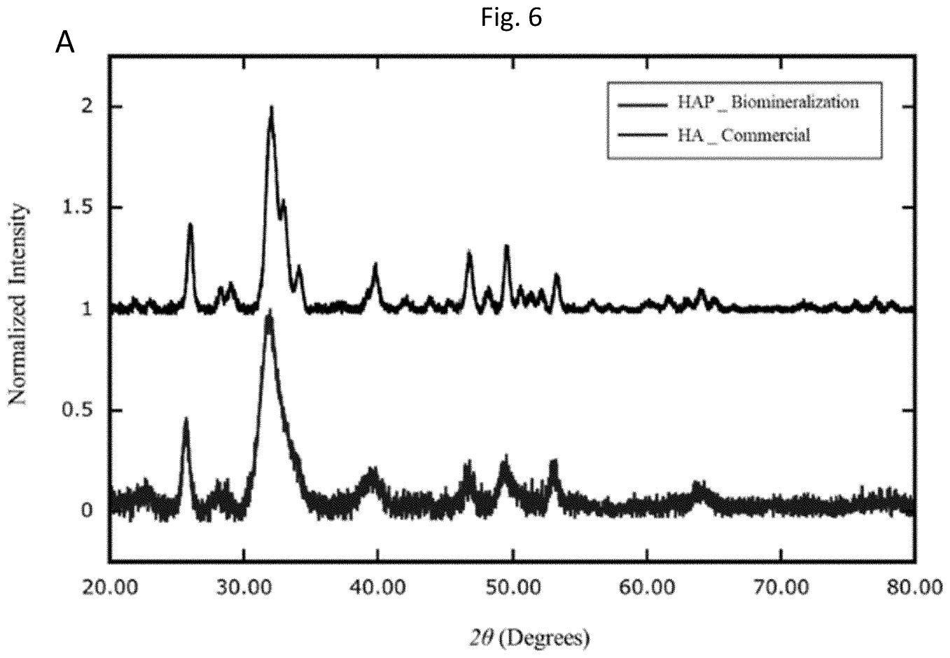

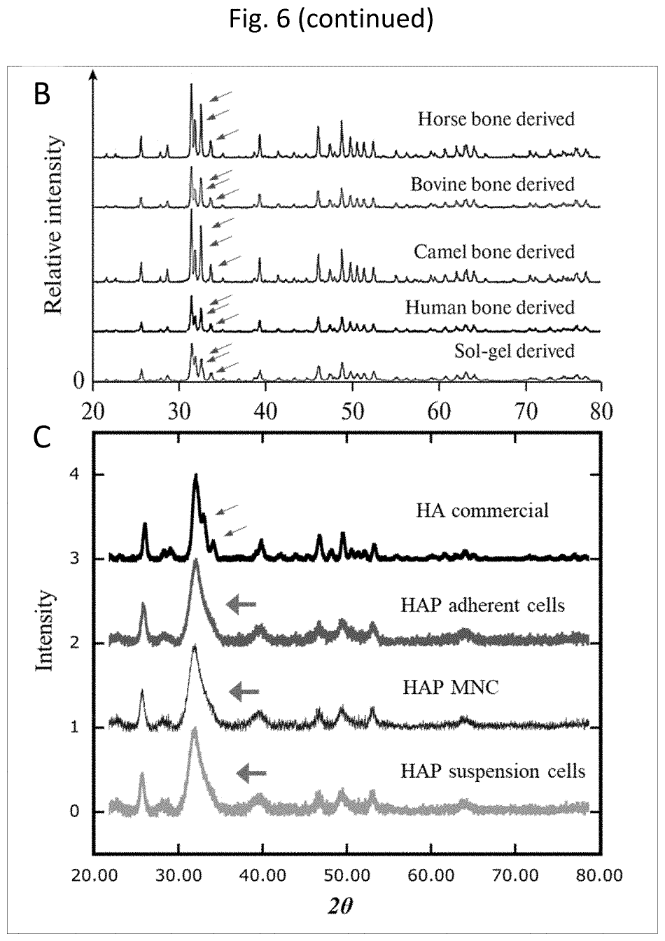

Natural HAP crystals produced by and/or collected by the methods of the present invention are also provided. Calcium phosphates are found in various phases (e.g., amorphous calcium phosphate and HAP) that differ in crystalline structure, composition, calcium/phosphate ratio, solubility, and bioresorbability. In preferred embodiments, the HAP of the present invention has a crystalline structure. As used herein, “crystalline structure” refers to a hexagonal structure of ions, molecules, or atoms that are held together in an ordered, three-dimensional arrangement. In the Examples, electron microscopy was used to characterize the HAP crystals that were produced in culture (FIG. 4). The inventors observed the progressive transformation of agglomerated amorphous calcium phosphate precursors into grains of HAP nano-crystallites. X-ray diffraction was used to confirm the mineral produced by these cells is genuine hydroxyapatite [Ca₁₀(PO₄)₆(OH)₂] (FIG. 6).

In some embodiments, the HAP crystallite particles are between 0.1 nm and 40 nm in size. The crystals of the present invention may have a size of at least 0.1 nm, 0.5 nm or 1 nm, but are no larger than 5 nm, 10 nm, 20 nm, or 40 nm, or any combination of ranges, e.g., 0.1-40 nm, 0.5-20 nm, or 1-10 nm. Bone is a composite material in which collagen fibrils form a scaffold for a highly organized arrangement of uniaxially oriented HAP crystals. Within bone, the HAP crystals are present in elongated plates or needles about 40 to 60 nm long, 20 nm wide, and 1.5 to 5 nm thick (Mate Sanchez de Val J E et al., (2016) Clin Oral Implants Res. 27(11):1331-1338). Thus, in preferred embodiments, the HAP crystals of the present invention have a particle size in the range of 5-10 nm. These crystals are substantially smaller than the bone void filler particles found in conventional ceramics and more closely mimic the native HAP found in bones and teeth. Importantly, the small size of the HAP particles produced by the methods of the present invention allows them to fit precisely within the space of collagen fibrils (˜40 nm), making them highly useful for the production of bone grafting materials.

### Biomedical Applications

The HAP provided by the present invention may be utilized in numerous biomedical applications. Calcium phosphates are commonly used in medicine and dentistry in the form of cements, coatings, scaffolds, and paste. HAP offers several advantages over other apatites in these applications due to its chemical similarity to the inorganic component of bone and tooth, making it particularly useful as a material for bone implants and dental prosthetics.

Thus, the present invention also encompasses methods of using the HAP produced by the methods disclosed herein to contact an object. In some embodiments, the object comprises collagen or pharmaceutical agent. For instance, HAP may be added to collagen to form a bone-like composition, which may be used in applications such as bone replacement by tissue engineering. With its ability to mask biomolecules and cross biological barriers, HAP has also proven useful as a drug delivery reagent. HAP has a low solubility under physiological pH conditions, which contributes to its slower degradation rate, allowing it to be used for controlled drug delivery by surgical placement or injection.

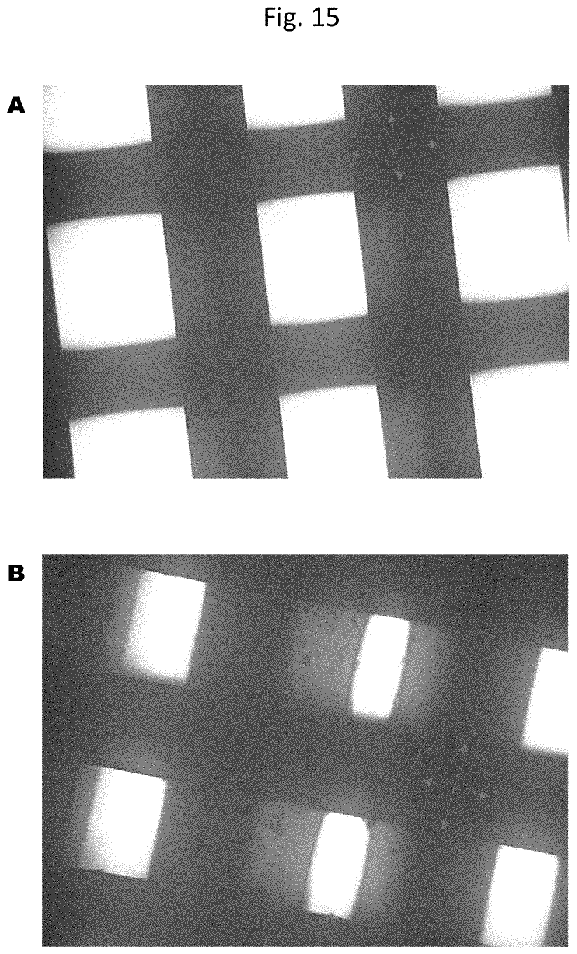

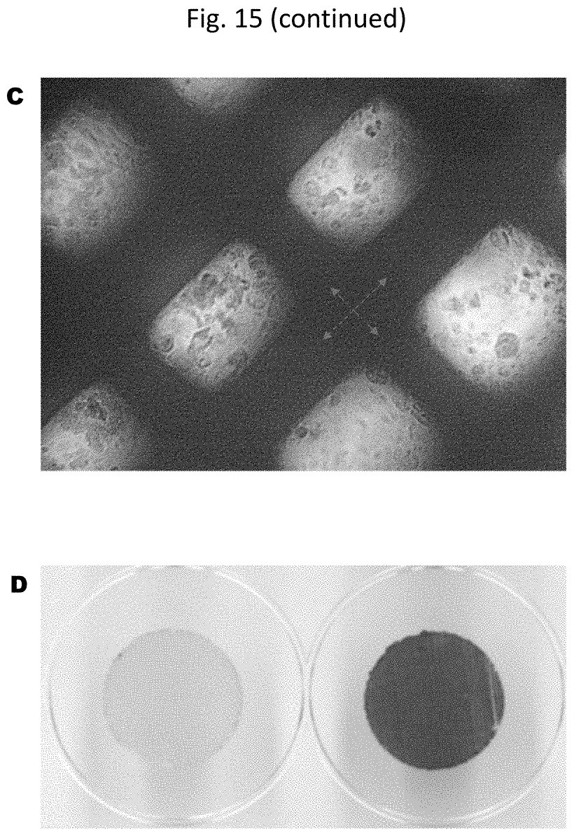

In other embodiments, the object contacted with HAP is a medical device, scaffold, or implant. For instance, HAP may be used to coat scaffolds designed to guide bone formation and neovascularization. Such scaffolds are useful for bone augmentations, artificial bone grafts, maxillofacial reconstruction, spinal fusion, periodontal disease repairs, and bone fillers after tumor surgery. Ultimately, to achieve biocompatibility and optimal mechanical properties, artificial scaffolds need to be coated or filled with autologous bone-like material. Often, at least 6 months are required for bone-tissue to replace the scaffold in the defect site. However, the efficiency of remodeling depends on several factors, including host anatomy and physiology, as well as the engineered tissue type. For example, in cancellous bone such remodeling takes about 3-6 months, whereas in cortical bone it will take roughly twice as long (i.e., about 6-12 months) [46]. In the Examples, the inventors demonstrate that poly ε-caprolactone scaffolds may be coated with HAP using the cell culture-based methods disclosed herein (FIG. 15) in as little as 20 days.

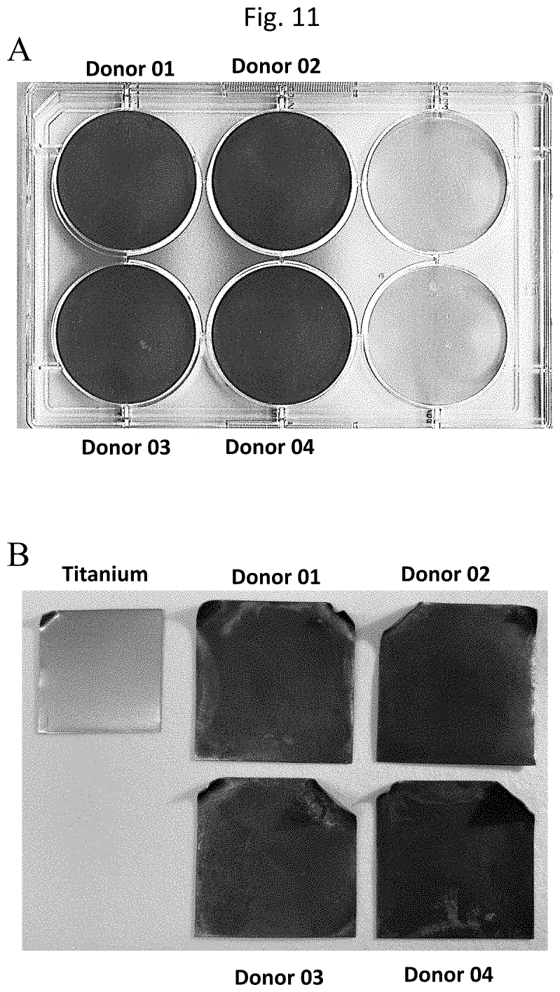

Further, using the biomineralization methods of the present invention, one may coat a variety of implant materials with HAP to improve their bioactivity, including metals, allograft bone particles, and structural scaffolds. In the Examples, the inventors demonstrate that titanium plates may be coated with HAP using the cell culture-based methods disclosed herein (FIG. 11).

### Methods of Measuring Organic Phosphates and Glycerophosphates

The present invention provides methods of measuring organic phosphates in a sample from a subject. The methods involve (a) obtaining a sample from the subject, (b) preparing a supernatant from the sample, (c) heat inactivating the supernatant, (d) incubating the supernatant of step c with alkaline phosphatase for at least 2 hours, and (e) performing a phosphorus detection assay and comparing the treated supernatant of step d with the heat inactivated supernatant of step c, wherein the difference equals the quantity of organic phosphorus in the sample.

Any phosphate detection assay may be utilized with these methods. Generally, such assays are used to detect the free inorganic phosphate present in a sample. In some embodiments, the phosphorus detection assay is a malachite green based assay. These assays rely on detection of a green complex formed between malachite green molybdate and free orthophosphate, which can be measured on a spectrophotometer (600-660 nm) or on a plate reader. Malachite green phosphate assay kits are commercially available (e.g., from Sigma-Aldrich).

The methods may be used to measure the presence of any organic phosphate in a sample from a subject. Organic phosphates, which are also known as organophosphates or phosphate esters, are a class of compounds that can be considered as esters of phosphoric acid. In some embodiments, the organic phosphate is a glycerophosphate (i.e., a phosphorylated glycerol).

Methods of measuring the glycerophosphates in a sample from a subject are also provided. These methods involve (a) obtaining a sample from the subject, (b) preparing a supernatant from the sample, (c) incubating the supernatant with a detectable substrate and a glycerophosphate oxidase, and (d) measuring the detectable substrate of the reaction of step c.

A glycerophosphate oxidase is an enzyme that catalyzes the oxidation of a glycerophosphate, releasing hydrogen peroxide (H₂O₂), which can be measured using a photometric assay. For Example, in FIG. 12, α-glycerophosphate (αGP) standards are subjected to a photometric assay catalyzed by horseradish peroxidase (HRP) to produce a standard curve. Thus, to quantify the αGP in a sample from a subject, the sample may be subjected to the same photometric assay and the amount of αGP present in the sample may be determined by comparing the results of the assay to this standard curve. However, any suitable detection assay may be utilized with the methods presented herein.

The detectable substrate may be directly or indirectly detectable, either by observation or by instrumentation. Detectable responses include, for example, colorimetry, fluorescence, chemiluminescence, phosphorescence, radiation from radioisotopes, magnetic attraction, and electron density. In certain embodiments, the detectable substrate is chromogenic or fluorogenic. Exemplary detectable substrates include, without limitation, horseradish peroxidase, 3,3′,5,5′-tetramethylbenzidine (TMB), 2,2′-Azinobis [3-ethylbenzothiazoline-6-sulfonic acid]-diammonium salt (ABTS), 0-phenylenediamine dihydrochloride (OPD) and 4-Aminoantipyrine.

While the most abundant supply of phosphates in mammals is the skeleton (i.e., in the form of HAP), phosphates are stored in tissues found throughout the body. Thus, the methods of the present invention may be performed on any tissue or fluid sample collected from a subject. Exemplary samples include urine, blood serum or plasma, cerebrospinal fluid, or bone marrow. While the inorganic phosphate found in serum reflects only a small percentage of total body phosphorus, it is easily measurable and is indicative of the general status of body phosphorus stores. Thus, in preferred embodiments, the sample is blood and the supernatant prepared in step b comprises serum.

As used herein, the term “supernatant” refers to the liquid lying above a solid residue after crystallization, precipitation, centrifugation, or another separation process. In some embodiments, the supernatant is heat inactivated. In certain embodiments in which the supernatant comprises serum, heating the supernatant inactivates serum complement (i.e., immune factors that may inhibit or destroy cells under certain conditions) to preserve the integrity of the subsequent assays. Heat inactivation is a known method in the art, and is commonly performed by heating the sample to at least 56° C. for at least 30 minutes. Preferably, the sample is heated to 60° C.-80° C.

### Kits for Measuring Glycerophosphates

In a final aspect, the present invention provides kits for measuring glycerophosphates in a sample from a subject. The kits comprise an oxidase, a glycerophoshate standard, and a detectable substrate capable of detecting hydrogen peroxide. The kits may also include additional materials that are useful for using the kits, such as additional reagents, buffers, and instruction manuals.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

Biomineralization is a trait of air-breathing vertebrates, in which an ossified skeleton is required to support the respiratory system. In biomineralization, mature osteoblasts and odontoblasts synthesize hydroxyapatite (HAP), which is deposited in the collagen matrix to construct endoskeleton. For many decades, researchers have studied the mechanisms that modulate differentiation and maturation of these specialized to gain insights into bone-remodeling defects.

In the following Example, the inventors demonstrate that biomineralization is a natural ability of cells cultured with three imperative elements: calcium, a phosphoester salt, and alkaline phosphatase (ALP). Calcium mineral deposition was observed in human cell lines derived from osteoblast, bone marrow stroma, embryo, muscle, and blood, from malignancies of breast, colon, prostate, cervix, leukemia, lymphoma, and myeloma, and in two mouse cell lines derived from fibroblast and myoblast. Surprisingly, biomineralization could also be induced in these cells using an ALP from other, non-human species. The biologically synthesized minerals were examined using electron microscopy, and found to comprise both amorphous calcium phosphate precursors and grains of nano-crystallites. X-ray diffraction analysis confirmed the mineral composition comprises genuine hydroxyapatite crystallites [Ca₁₀(PO₄)₆(OH)₂] with a typical size of 5-10 nanometers.

### Materials and Methods:

Cell lines: The following mammalian cell lines were obtained from the American Type Culture Collection: human osteoblast hFOB1.19; human osteosarcoma MG-63 and Saos-2; human bone marrow stroma HS-5; human embryonic kidney HEK-293; human rhabdomyosarcoma RD; human mammary gland adenocarcinoma MDA-MB-231 and MCF-7; human colorectal adenocarcinoma SW480, SW620, COLO 205, and COLO 32DM; human prostate carcinoma DU145, LNCaP FGC, and PC-3; human cervical adenocarcinoma HeLa; human leukemia HL-60, K562, and THP1; human lymphoma U937; human myeloma plasma cells HCl-H929, OPM2, RPMI8226, and U266; mouse fibroblast NIH/3T3; and mouse myoblast C2C12. Human myeloma cell lines ARK, ARP1, and CAG were developed in-house. The JJN3 myeloma cell line was provided by Michael Kuehl, MD (National Cancer Institute, Bethesda, Md., USA). Human peripheral blood mononuclear cells were obtained from ZENBIO (Research Triangle Park, N.C., USA). All cell lines were cultured in minimum essential medium alpha containing 10% fetal bovine serum (MEMα/10% FBS), penicillin/streptomycin (50 μg/mL of each), and L-glutamine (2 mM; see Table 1 for formulation).

Materials: The following were purchased from ThermoFisher Scientific (Carlsbad, Calif., USA): MEMα with or without phenol red; MEMα with or without ascorbic acid (vitamin C); MEMα with or without calcium (Ca²⁺); penicillin/streptomycin; L-glutamine; TaqMan gene expression assays for ALPG (Hs00741068_g1), ALPI (Hs00357579_g1), ALPL (Hs10129144_m1), ALPP (Hs00740632_gH), GAPDH (Hs99999905_m1); universal PCR master mix. Recombinant human ALPL was purchased from R&D Systems (Minneapolis, Minn., USA). Rabbit anti-human ALPL antibody, calf intestinal ALP (CIP, ≥10 DEA U/mg), disodium β-glycerophosphate (βGP), phospho(enol)pyruvate monosodium (PEP), pamidronate, dexamethasone, and Alizarin Red S solution were purchased from Sigma-Aldrich (St. Louis, Mo., USA). CIP (≥10,000 U/mL), shrimp hepatopancreas ALP (≥1,000 unit/mL), and p-nitrophenyl phosphate kits were purchased from New England BioLabs (Ipswich, Mass., USA). FBS was purchased from Atlanta Biologicals (Flowery Branch, Ga., USA). Disodium α-glycerophosphate hydrate (1 M of αGP; Glycophos) was purchased from Fresenius Kabi (Lake Zurich, Ill., USA), and glycerophosphoric acid (NSC 9231) was obtained from the Developmental Therapeutics Program (NCI, USA). Collagen Type I, rat tail, stock solution was obtained from BD Biosciences Discovery Labware (Waltham, Mass., USA).

Quantitative RT-PCR and western immunoblot detection: Total RNA was extracted from cells with an RNeasy Plus mini kit (Qiagen, Hilden, Germany). Reverse transcription was carried out with 500 ng of total RNA with the SuperScript III first-strand system with random hexamer primers, according to the manufacturer's instructions (ThermoFisher Scientific). cDNA derived from 10 ng of total RNA was used for gene expression assays in TaqMan real-time PCR (20 μL reaction mix); TaqMan assays ran 40 thermocycles for amplification. Quantitative expression of ALPG, ALPI, ALPL, or ALPP gene was calculated based on ΔΔCT relative to GAPDH expression. For protein electrophoresis, each lane was loaded with 20 μg of cell lysate in RIPA lysis buffer (Santa Cruz Biotechnology, Dallas, Tex., USA). After electro-blotting to PVDF membrane, a WesternBreeze kit was used for immunodetection with antibodies to human ALPL and GAPDH (Santa Cruz Biotechnology, Calif., USA).

Biomineralization assays and Alizarin Red S staining: All human and mouse cell lines and human peripheral blood mononuclear cells (MNCs) were maintained in MEMα/10% FBS with penicillin/streptomycin and glutamine in a 37° C. humidified incubator with 5% CO₂. In biomineralization assays, the concentrations of αGP (or (3GP, NSC 9132, pamidronate, or PEP) as the primary source of organic phosphorus was standardized at 2 mM; and ALPL, CIP, or SAP as a primary source of ALP was standardized at 1 unit/mL (U/mL), respectively. Ascorbic acid (vitamin C) was added at 50 μg/mL (0.284 mM). Dexamethasone (Dex) was added at 100 nM. For adherent cell lines, the assays were performed after 70% confluence was reached in 24-, 12-, or 6-well plates (ThermoFisher Scientific). For nonadherent cell lines, assays were performed in triplicate at an initial density of 1×10⁴ cell/well in 96-well plates (ThermoFisher Scientific). The medium was replaced after 4 days. Alizarin Red S staining was performed at room temperature by removing the culture medium, washing the cells twice with 1×PBS (pH 7.4), fixing the cells in 10% neutral buffered formalin (Richard-Allan Scientific, Kalamazoo, Mich., USA) for 30 min, rinsing twice with Milli-Q water, staining with Alizarin Red S for 30 min, and destaining twice in Milli-Q water. A ZEISS inverted fluorescence/brightfield microscope equipped with an Infinity 3 digital camera and software system was used for imaging.

Coating cell culture plate with Collagen Type I, rat tail for mineralization assays: To coat a 48-well plate with Collagen Type I at 5 μg/cm², the stock solution was diluted to 25 μg/mL in 17.5 mM of acetic acid and aliquoted 0.8 mL into each well. After incubating at room temperature for one hour, the Collagen Type I solution was removed. Each well was rinsed with 1×PBS (pH 7.4) and air-dried at room temperature. Human blood MNCs were suspended in the conditioned media and distributed into each well (1×10⁵ cell/well) prior to a 7-day incubation.

The media were changed on day 4.

Hydroxyapatite purification for X-ray diffraction and electron microscopy: The precipitated minerals were collected by scraping, washed twice with 1×PBS (pH 7.4), and suspended in sodium hydroxide (10%) for 30 min. Mineral content was extracted twice with acetone or chloroform and washed twice with 100% ethanol. For X-ray diffraction, purified minerals were air-dried in a spin-vacuum for 30 min at 60° C. The crystallinity, size, texture, and homogeneity of the dry powder were analyzed with a Bruker D8-Discover X-Ray Diffractometer. For high-resolution transmission electron microscopy, the mineral suspension (in 100% ethanol) was ground manually in a glass grinder and dropped onto a carbon-coated copper grid (Sigma-Aldrich) to allow the ethanol to evaporate. The ultrastructure was examined under a transmission electron microscope (FEI Tecnai F20 200 keV, JEM-2100F, or FEI Titan 80-3000) equipped with a field emission gun (0.1-nm lattice resolution).

TABLE 1
Formulation of minimum essential medium
alpha (MEMα) with nucleosides.

| | | | |
|---|---|---|---|
| | Molecular | Concentration | |
| Component | weight | (mg/L) | mM |

Amino Acids

| | | | |
|---|---|---|---|
| Glycine | 75.0 | 50.0 | 0.6666667 |
| L-Alanine | 89.0 | 25.0 | 0.28089887 |
| L-Arginine hydrochloride | 211.0 | 105.0 | 0.49763033 |
| L-Asparagine-H2O | 150.0 | 50.0 | 0.33333334 |
| L-Aspartic acid | 133.0 | 30.0 | 0.22556391 |
| L-Cysteine | 176.0 | 100.0 | 0.5681818 |
| hydrochloride-H2O | | | |
| L-Cystine 2HCl | 313.0 | 31.0 | 0.09904154 |
| L-Glutamic Acid | 147.0 | 75.0 | 0.5102041 |
| L-Glutamine | 146.0 | 292.0 | 2.0 |
| L-Histidine | 155.0 | 31.0 | 0.2 |
| L-Isoleucine | 131.0 | 52.4 | 0.4 |
| L-Leucine | 131.0 | 52.0 | 0.39694658 |
| L-Lysine | 183.0 | 73.0 | 0.3989071 |
| L-Methionine | 149.0 | 15.0 | 0.10067114 |
| L-Phenylalanine | 165.0 | 32.0 | 0.19393939 |
| L-Proline | 115.0 | 40.0 | 0.3478261 |
| L-Serine | 105.0 | 25.0 | 0.23809524 |
| L-Threonine | 119.0 | 48.0 | 0.40336135 |
| L-Tryptophan | 204.0 | 10.0 | 0.04901961 |
| L-Tyrosine disodium salt | 225.0 | 52.0 | 0.23111111 |
| L-Valine | 117.0 | 46.0 | 0.3931624 |

Vitamins

| | | | |
|---|---|---|---|
| Ascorbic Acid | 176.0 | 50.0 | 0.2840909 |
| Biotin | 244.0 | 0.1 | 4.0983607E−4 |
| Choline chloride | 140.0 | 1.0 | 0.007142857 |
| D-Calcium pantothenate | 477.0 | 1.0 | 0.002096436 |
| Folic Acid | 441.0 | 1.0 | 0.0022675737 |
| Niacinamide | 122.0 | 1.0 | 0.008196721 |
| Pyridoxal hydrochloride | 204.0 | 1.0 | 0.004901961 |
| Riboflavin | 376.0 | 0.1 | 2.6595744E−4 |
| Thiamine hydrochloride | 337.0 | 1.0 | 0.002967359 |
| Vitamin B12 | 1355.0 | 1.36 | 0.0010036901 |
| i-Inositol | 180.0 | 2.0 | 0.011111111 |

Inorganic Salts

| | | | |
|---|---|---|---|
| Calcium Chloride | 111.0 | 200.0 | 1.8018018 |
| (CaCl2) (anhyd.) | | | |
| Magnesium Sulfate | 120.0 | 97.67 | 0.8139166 |
| (MgSO4) (anhyd.) | | | |
| Potassium Chloride | 75.0 | 400.0 | 5.3333335 |
| (KCl) | | | |
| Sodium Bicarbonate | 84.0 | 2200.0 | 26.190475 |
| (NaHCO3) | | | |
| Sodium Chloride (NaCl) | 58.0 | 6800.0 | 117.24138 |
| Sodium Phosphate | 138.0 | 140.0 | 1.0144928 |
| monobasic | | | |
| (NaH2PO4—H2O) | | | |

Ribonucleosides

| | | | |
|---|---|---|---|
| Adenosine | 267.0 | 10.0 | 0.037453182 |
| Cytidine | 243.0 | 10.0 | 0.041152265 |
| Guanosine | 283.0 | 10.0 | 0.03533569 |
| Uridine | 244.0 | 10.0 | 0.040983606 |

Deoxyribonucleosides

| | | | |
|---|---|---|---|
| 2′Deoxyadenosine | 251.0 | 10.0 | 0.03984064 |
| 2′Deoxycytidine HCl | 264.0 | 11.0 | 0.041666668 |
| 2′Deoxyguanosine | 267.0 | 10.0 | 0.037453182 |
| Thymidine | 242.0 | 10.0 | 0.041322313 |

Other Components

| | | | |
|---|---|---|---|
| D-Glucose (Dextrose) | 180.0 | 1000.0 | 5.5555553 |
| Lipoic Acid | 206.0 | 0.2 | 9.708738E−4 |
| Phenol Red | 376.4 | 10.0 | 0.026567481 |
| Sodium Pyruvate | 110.0 | 110.0 | 1.0 |

### Results:

### The Essential Elements for Biomineralization

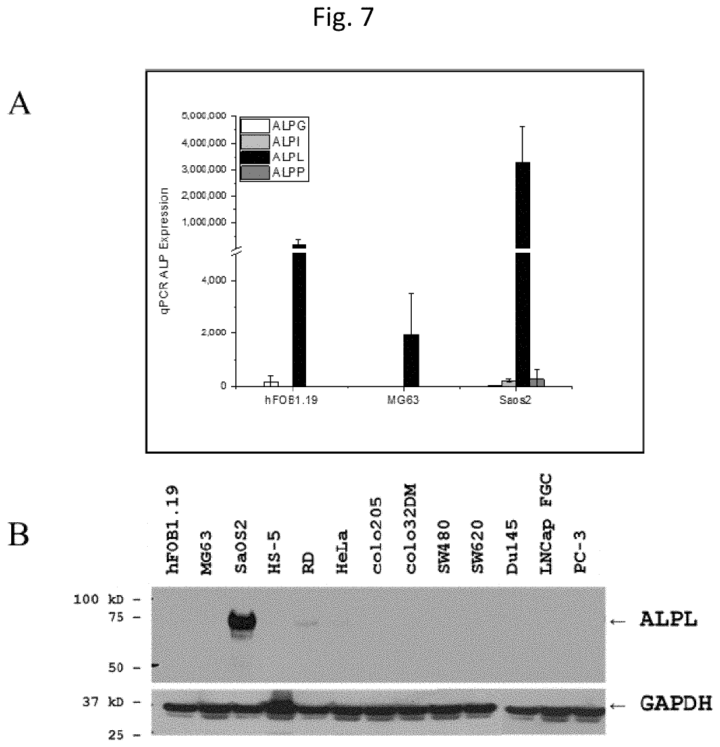

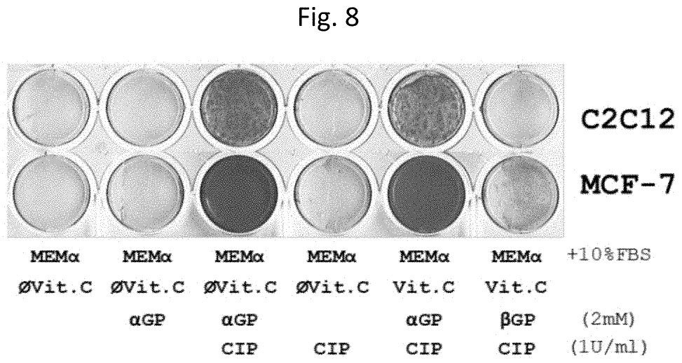

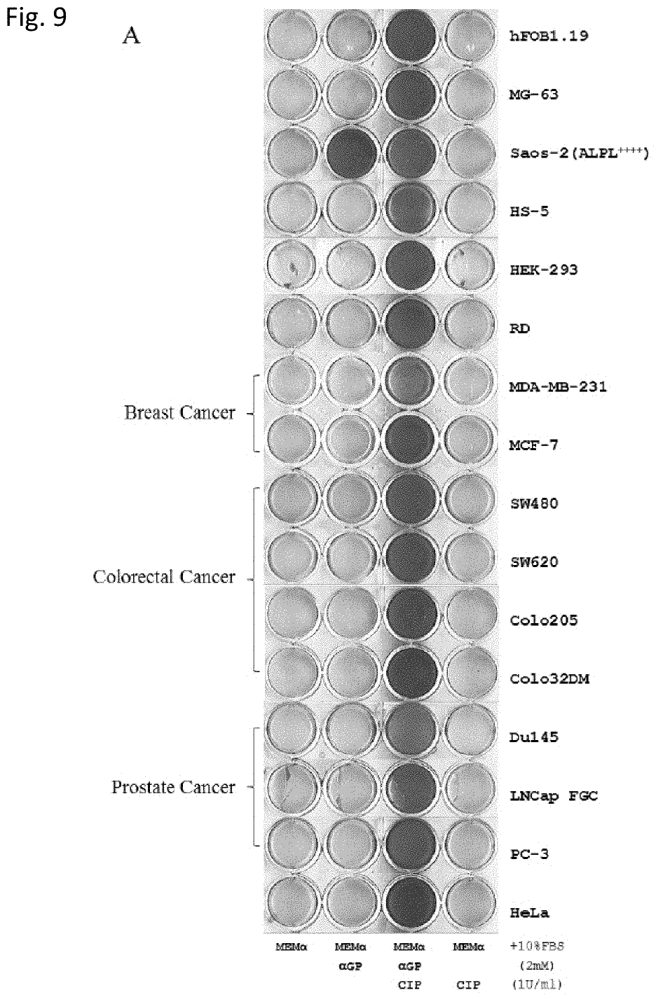

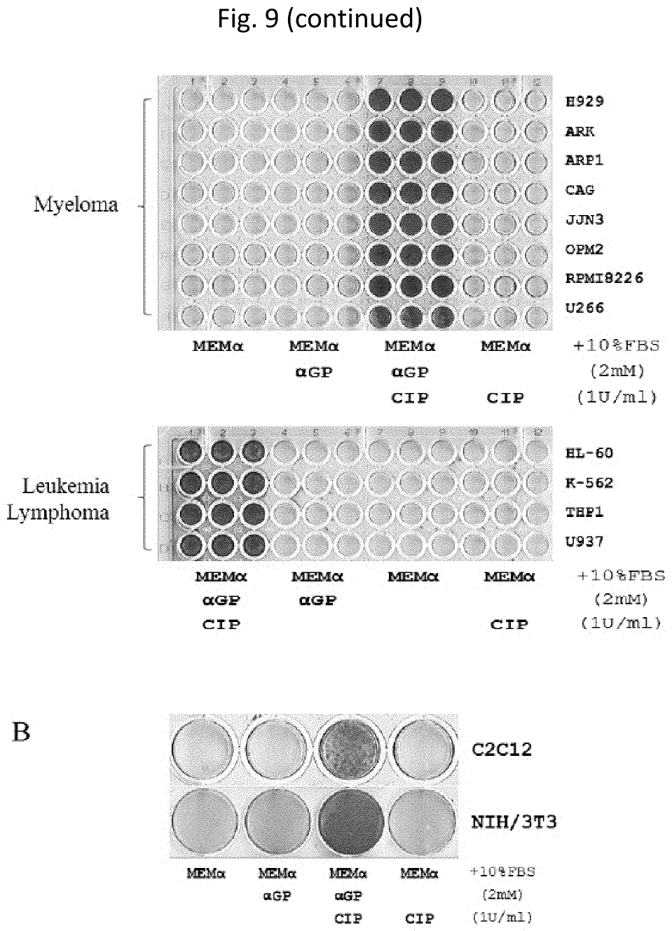

We used Alizarin Red S (ARS) staining assays to investigate biomineralization in two human osteosarcoma cell lines, Saos-2 and MG-63. Under different culture conditions, we observed that Saos-2 cell line could mineralize within seven days of culture in minimum essential medium alpha with 10% FBS (MEMα/10% FBS) supplemented only with disodium β-glycerophosphate (βGP), but MG-63 could not (FIG. 1A). Similar results were observed after 21-28 days of incubations (images are not shown). Gene expression profiling and western blot analysis determined that Saos-2 cells expressed high levels of tissue-nonspecific alkaline phosphatase (ALPL) (FIG. 7). To explore the role of ALPL in biomineralization, recombinant human ALPL and βGP were added to MG-63 and Saos-2 cell lines cultured in MEMα/10% FBS; within seven days, biomineralization was observed in both cell lines (FIG. 1A), suggesting that such reaction requires concomitant presences of ALPL and βGP in MEMα, which contains 1.8 mM calcium. Notably, similar results reiterated when human ALPL was substituted by alkaline phosphatase from calf intestine (CIP) or from shrimp hepatopancreas (SAP) (FIG. 1A) and when βGP was substituted with either disodium α-glycerophosphate (αGP) or sodium phospho(enol)pyruvate (PEP) (FIG. 1B,C). When bisphosphonate (pamidronate) or glycerophosphoric acid (NSC 9231) was supplemented as an organic phosphate source, biomineralization did not occur (FIG. 1C). Further, biomineralization was performed well without ascorbic acid (Vit. C), suggesting that it may be unnecessary or even deleterious to the process (FIG. 1A and FIG. 8). The titration assays indicated that biomineralization was dose-dependent on αGP, ALP, and calcium (FIG. 1E-G), and the reaction did not occur if any one of the three elements was missing (FIG. 1, FIG. 8, and FIG. 9).

### Biomineralization is an Innate Ability of all Mammalian Cells

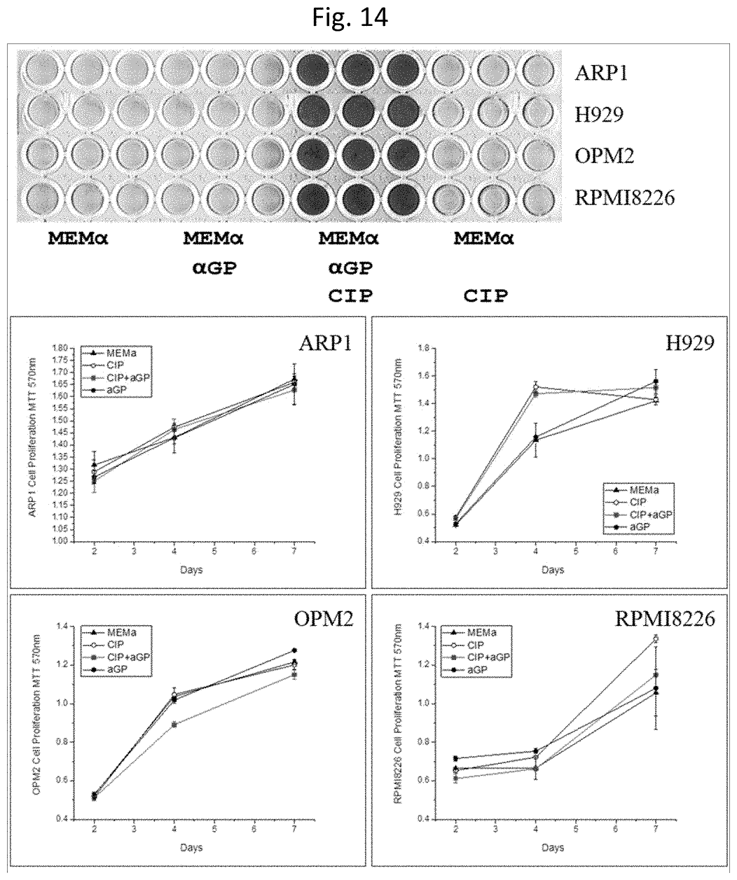

In addition to MG-63 and Saos-2 cell lines, we investigated biomineralization in other mammalian cells under similar conditions and without inducing differentiation. These included 26 human cell lines derived from osteoblast, bone marrow stroma, embryo, muscle, breast cancer, colon cancer, prostate cancer, cervical carcinoma, leukemia, lymphoma, multiple myeloma (FIG. 9A), undifferentiated human mononuclear cells (MNCs) from peripheral blood (FIG. 1D); and two mouse cell lines (FIG. 9B). Within seven days, ARS demonstrated that a wide variety of human and mouse cells have the innate ability of self-mineralization. Without living cells, biomineralization reaction did not occur in the cell-less wells that were coated with Collagen Type I (rat tail) and containing MEMα/10% FBS, αGP, and ALP after seven-day incubation (FIG. 1H). Importantly, the viability and proliferation of cells undergoing biomineralization were not disrupted by the process (FIG. 14).

### The Pathway of Biomineralization

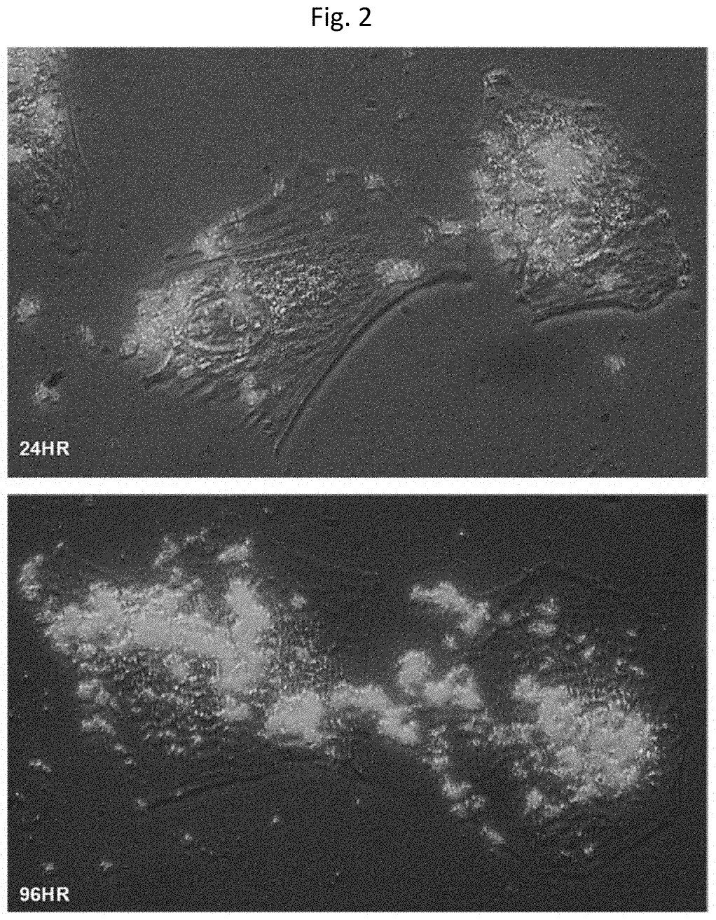

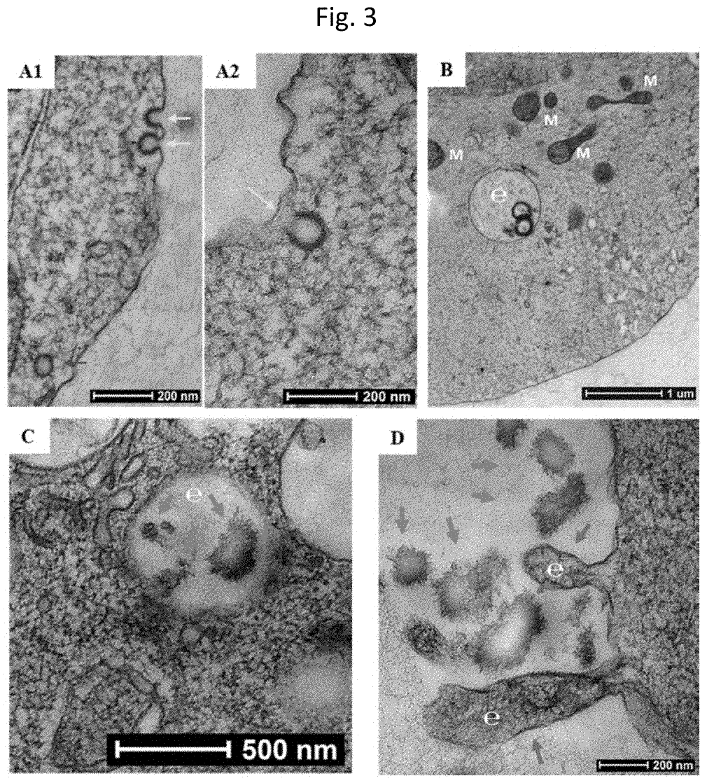

To characterize the process of biomineralization, we monitored the mineral formation in Saos-2 cells cultured in MEMα/10% FBS supplemented with 2 mM of αGP. ARS staining demonstrated that biomineralization occurred at cytoplasmic membrane and cytosol of the adherent cells within 24 h of incubation. A large amount of intracellular accumulation and extracellular secretion of HAP were observed after 96 h of incubation (FIG. 2). Further, we investigated this biological process using high-resolution electron microscopy. A non-adherent leukemia cell line, K562, was cultured for 72 h in MEMα/10% FBS supplemented with αGP and CIP. In preparation for transmission electron microscopy (TEM), cell morphology was preserved with high-pressure freezing and freeze-substitution [33]. Serial sections of the cell ultrastructure revealed that the formation of mineral caveolae at the cytoplasmic membrane was the first step of biomineralization (FIG. 3A1,A2). Endocytosis of caveolae transported the mineral matrixes into endosomes (FIG. 3B), where the calcium phosphate agglomerates were constructed (FIG. 3C). Eventually, the mineral agglomerates were carried to the membrane by endosomes and then released into the extracellular space (FIG. 3D).

### Crystallinity and Atomic Composition of the Mineral Agglomerates

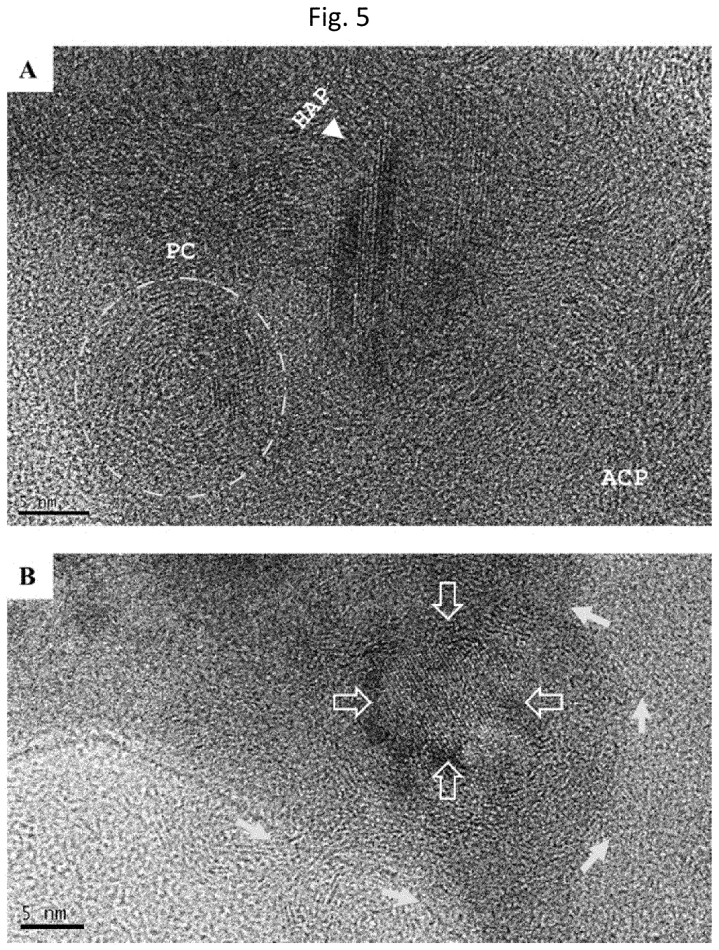

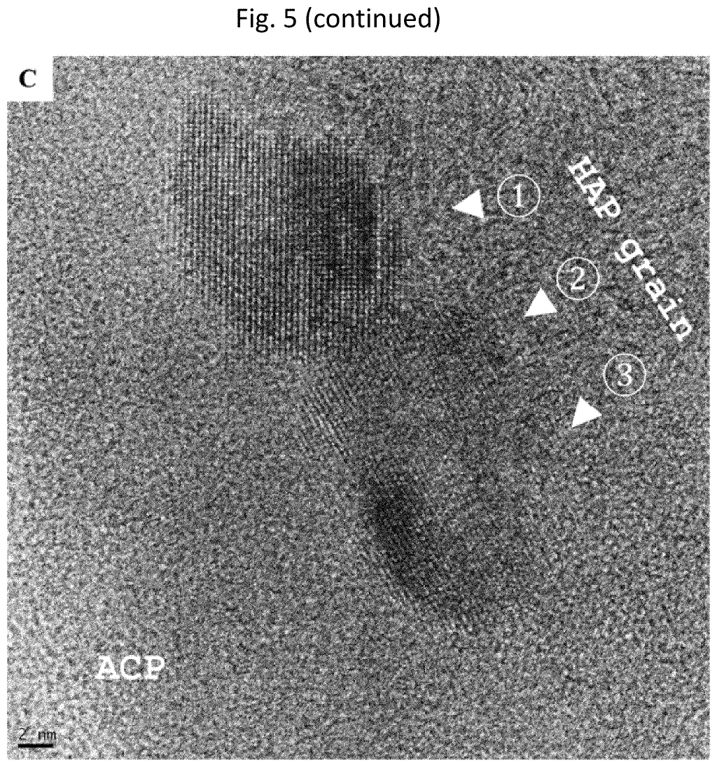

Biologically generated mineral agglomerates from Saos-2, MG-63, MCF-7, PC-3, K562, and RPMI8226 cell lines and MNCs were purified and examined under high-resolution TEM. Unlike chemically synthetized large (>50 nm) spherical particles ([9] and (FIG. 4A), the agglomerates from biomineralization were composed of small granules containing stochastic amorphous calcium phosphate (ACP) (FIG. 4B-D), which was ultimately transformed into crystalline HAP. Before crystallization, ACP coiling occurred as a precrystalline stage that aggregated ACP into a polycrystalline mass (“onion ring” in FIG. 5A). The primary transformation occurred at the center of coiled ACP with an explicit crystallographic texture of HAP in sizes of 5-10 nm (FIG. 5B). This primary event triggered a chain reaction that expanded HAP to crystallite grains (FIG. 5C). The formation of grain boundaries (FIG. 4E) indicated that the agglomerates could further disintegrate into thin films of HAP grains, with an approximate thickness of 5-10 nm fit into the spaces (˜40 nm) between collagen fibrils in bone 34,35].

The composition and crystallinity of the biologically generated minerals were analyzed with an X-ray diffractometer [33,36]. The atomic composition of the extracted nanocrystals was genuine HAP, Ca₁₀(PO₄)₆(OH)₂; the atomic composition of the commercial bony nanoparticles was identified as calcium phosphate hydrates, Ca₃(PO₄)₂·xH₂O (FIG. 6A). In contrast, artificial bony ceramics and recovered bone ores are heterogeneous calcium phosphate hydrate of Ca₃(PO₄)₂·xH₂O, which comprise multiple structures represented by stacking faults along with the peak of authentic HAP (indicated by red arrows in FIG. 6B-6C).

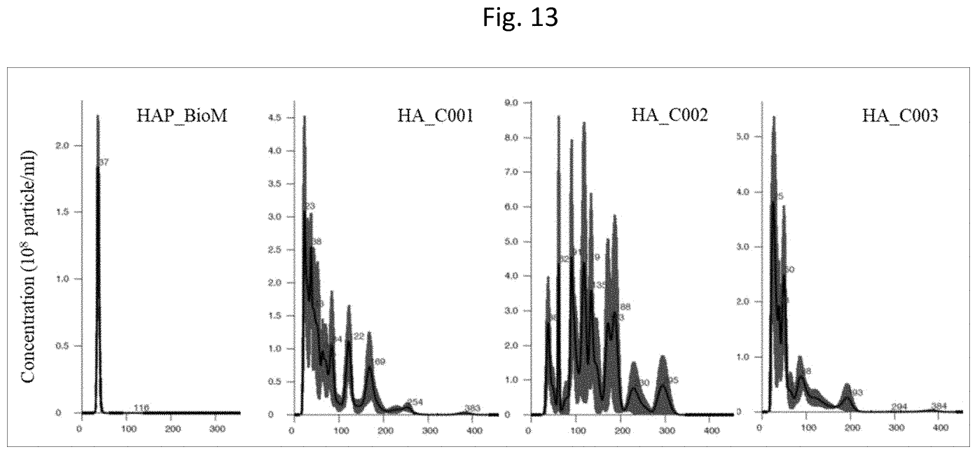

Additionally, the biologically synthetized HAP particles were measured to have an average size of 36.4±3.1 nm by nanoparticle tracking analysis (NTA). This instrument traces the size distribution and concentration of particles based on their Brownian motion. FIG. 13 shows that the NTA profile of natural HAP (BioM) is significantly different from artificial HAP nanomaterials (HA_C001, HA_C002, and HA_C003), which are characterized by a wide variety of physical sizes. This small particle size of the biologically synthetized HAP represents a significant advantage, allowing the particles to fill in the 40 nm spaces between collagen fibrils.

Human Mononuclear Cells are Capable of Biomineralization without Prior Cellular Differentiation

Human mononuclear cells (MNC) were isolated from the peripheral blood of healthy adult donors by removing erythrocytes and serum. In a 6-well TC-plate, 5 million MNC were seeded in MEMα/10% FBS containing αGP (2 mM) and CIP (1 U/ml) and incubated for 7 days. In a separate TC plate, 5 million MNC were seeded in the same medium on top of a piece of titanium foil and incubated for 14 days. The media was refreshed every 4 days. Alizarin Red S staining was performed by the end of each assay. Non-adherent MNC were collected by centrifugation in each staining step. On day 7, significant calcium phosphate mineral staining was present in all tested wells (FIG. 11A). By day 14, the pieces of titanium foil were coated with minerals secreted by the MNC (FIG. 11B).

In another experiment, the MNC were seeded on top of poly ε-caprolactone (PCL) scaffold (MilliporeSigma Co). After a 20 day incubation, the scaffold was coated with HAP and the spaces within the scaffold were partially filled with HAP, as indicated by Alizarin Red S staining (FIG. 15).

### Discussion

Our experiments indicate that ALP from hierarchically distant species (human, bovine, and shrimp) can function as isozymes (FIG. 1, FIG. 8, and FIG. 9), despite substantial differences in their primary structures (FIG. 10). Recently, controlled HAP biomineralization was identified in ˜810 million-year-old fossils of primitive eukaryotes [32]. The earliest known vertebrate came into existence 300 million years later, so the function of HAP in these early unicellular organisms is not clear. In present-day invertebrates, which lack an internal skeleton, HAP formation has been observed on the mandibular teeth of most crustaceans, suggesting that biomineralization is widely conserved across Kingdom Animalia [37-39]. Together, these recent findings and our results suggest that the controlled HAP biomineralization is the result of an innate ability of Eukarya that underlies their evolution and survival.

Phosphorus is a key element of bone and a core component of buffer systems that maintain pH homeostasis in the body. Based on our current results, αGP is one of the most efficient acyclic alkane (CₙH₂ₙ₊₂) phosphoester salts for promoting biomineralization (FIG. 1, FIG. 8, and FIG. 9). In eukaryotes, αGP is an intermediate metabolite of lipid metabolism that contributes to the mitochondrial electron transport chain [40]. In prokaryotes, which lack mitochondria, lipid metabolism occurs in the cytosol to release αGP to the extracellular space [37,38]. In bacteria-animal symbioses, the host eukaryotic cells can “outsource” the αGP produced by prokaryotes to sustain their biological activities [41,42].

The human genome contains four ALP genes: intestinal alkaline phosphatase (ALPI), germ-cell alkaline phosphatase (ALPG), placental alkaline phosphatase (ALPP), and ALPL. ALPI, ALPG, and ALPP are generally inactive (FIG. 7). ALPL is present at high levels in bone, liver, kidney, brain, skin, and vascular endothelial cells [26], which are typical locations of cancer metastasis and sites of ectopic calcification. Although ALPs function primarily to catalyze the hydrolysis of phosphoric ester from organic compounds known as dephosphorylation under basic pH conditions, the precise functions of these isozymes at more acidic and physiological conditions are poorly understood. A century ago, Robert Robison, PhD (1883-1941), discovered that a phosphoric esterase (i.e., ALP) is essential for bone mineralization [43-45]. Our study repeatedly demonstrated that innate biomineralization could not be achieved when mammalian cells exposed to MEMα comprised of Ca′, inorganic phosphate (1.01 M of NaH₂PO₄), and FBS within seven days, even by adding βGP or αGP; unless an ALP was also present (FIG. 1, FIG. 8, and FIG. 9). Hence, our study highlights the crucial function of ALP in control of biomineralization.

### Conclusions:

Our study clearly indicates that biomineralization is an innate ability of any given somatic cell and requires the concomitant presence of three indispensable elements: Ca′, a phosphoester salt, and an ALP isozyme. We propose that bone regeneration and ectopic calcification are governed by the local balance of these three factors.

### REFERENCES

1. Andersen, T. L.; Sondergaard, T. E.; Skorzynska, K. E.; Dagnaes-Hansen, F.; Plesner, T. L.; Hauge, E. M.; Plesner, T.; Delaisse, J. M. A Physical Mechanism for Coupling Bone Resorption and Formation in Adult Human Bone. Am. J. Pathol. 2009, 174, 239-247.
2. Raggatt, L. J.; Partridge, N. C. Cellular and Molecular Mechanisms of Bone Remodeling. J Biol. Chem. 2010, 285, 25103-25108.
3. Kong, Y. Y.; Yoshida, H.; Sarosi, I.; Tan, H. L.; Timms, E.; Capparelli, C.; Morony, S.; Oliveira-dos-Santos, A. J.; Itie, A.; Khoo, W.; et al. OPGL is a key regulator of osteoclastogenesis, lymphocyte development and lymph-node organogenesis. Nature 1999, 397, 315-323.
4. Hofbauer, L. C.; Khosla, S.; Dunstan, C. R.; Lacey, D. L.; Boyle, W. J.; Riggs, B. L. The roles of osteoprotegerin and osteoprotegerin ligand in the paracrine regulation of bone resorption. J. Bone Miner. Res. 2000, 15, 2-12.
5. Teitelbaum, S. L.; Ross, F. P. Genetic regulation of osteoclast development and function. Nat. Rev. Genet. 2003, 4, 638-649.
6. Ducy, P.; Schinke, T.; Karsenty, G. The Osteoblast: A Sophisticated Fibroblast under Central Surveillance. Science 2000, 289, 1501-1504.
7. Bruderer, M.; Richards, R. G.; Alini, M.; Stoddart, M. J. Role and regulation of Runx2 in osteogenesis. Eur. Cells Mater. 2014, 28, 26-86.
8. Martin, T. J. Historically significant events in the discovery of RANK/RANKL/OPG. World J. Orthop. 2013, 4, 186-197.
9. Blair, H. C.; Larrouture, Q. C.; Li, Y.; Lin, H.; Beer-Stoltz, D.; Liu, L.; Tuan, R. S.; Schlesinger, R. L. J.; Nelson, D. J. Osteoblast differentiation and bone matrix formation in vivo and in vitro. Tissue Eng. 2017, 23, 268-280.
10. Eghbali-Fatourechi, G. Z.; Lamsam, J.; Fraser, D.; Nagel, D.; Riggs, B. L.; Khosla, S. Circulating osteoblast-lineage cells in humans. N. Engl. J. Med. 2005, 352, 1959-1966.
11. Esteve, F. R.; Roodman, G. D. Pathophysiology of myeloma bone disease. Best Pract. Res. Clin. Haematol. 2007, 20, 613-624.
12. Weiner, S.; Dove, P. M. An Overview of Biomineralization Processes and the Problem of the Vital Effect. Rev. Mineral. Geochem. 2003, 54, 1-29.
13. Knoll, A. H. Biomineralization and Evolutionary History. Mineral. Soc. Am. 2003, 54, 329-350.
14. Friedenstein, A. J.; Chailakhyan, R. K.; Gerasimov, U. V. Bone marrow osteogenic stem cells: In vitro cultivation and transplantation in diffusion chambers. Cell Tissue Kinet. 1987, 20, 263-272.
15. DiGirolamo, C. M.; Stokes, D.; Colter, D.; Phinney, D. G.; Class, R.; Prockop, D. J. Propagation and senescence of human marrow stromal cells in culture: A simple colony-forming assay identifies samples with the greatest potential to propagate and differentiate. Br. J. Haematol. 1999, 107, 275-281.
16. Arkady Rutkovskiy, A.; Stenslokken, K.; Vaage, I. J. Osteoblast differentiation at a glance. Med Sci. Monit. Basic Res. 2016, 22, 95-106.
17. Ducy, P.; Schinke, T.; Karsenty, G. The Osteoblast: A Sophisticated Fibroblast under Central Surveillance. Science 2000, 289, 1501-1504.
18. Ducy, P.; Desbois, C.; Boyce, B.; Pinero, G.; Story, B.; Dunstan, C.; Smith, E.; Bonadio, J.; Goldstein, S.; Gundberg, C.; et al. Increased bone formation in osteocalcin-deficient mice. Nature 1996, 382, 448-452.
19. Denu, R. A.; Nemcek, S.; Bloom, D. D.; Goodrich, A. D.; Kim, J.; Mosher, D. F.; Hematti, P. Fibroblasts and Mesenchymal Stromal/Stem Cells Are Phenotypically Indistinguishable. Acta Haematol. 2016, 136, 85-97.
20. Chung, C. H.; Golub, E. E.; Forbes, E.; Tokuoka, T.; Shapiro, I. M. Mechanism of action of 3-glycerophosphate on bone cell mineralization. Calcif. Tissue Int. 1992, 51, 305-311.
21. Gregory, C. A.; Gunn, W. G.; Peister, A.; Prockop, D. J. An Alizarin red-based assay of mineralization by adherent cells in culture: Comparison with cetylpyridinium chloride extraction. Anal. Biochem. 2004, 329, 77-84.
22. Zangari M, Yaccoby S, Pappas L, Cavallo F, Kumar N S, Ranganathan S, Suva L J, Gruenwald J M, Kern S, Zhan F, Esseltine D, Tricot G. A prospective evaluation of the biochemical, metabolic, hormonal, and structural bone changes associated with bortezomib response in multiple myeloma patients. Haematologica 2011, 96: 333-6
23. Esteve, F. R. Roodman, G. D. Pathophysiology of myeloma bone disease. Best Pract Res Clin Haematol. 2007, 20: 613-624
24. Nayak, A. K. Hydroxyapatite Synthesis Methodologies: An Overview. Int J of ChemTech Res 2010, 2: 903-07
25. Tian, E. Watanabe, F. Martin, B. Zangari, M. Innate Biomineralization. Int. J Mol. Sciences 2020, 21: 4820-34
26. Weiss, M. J. Henthorn, P. S. Lafferty, M. A.; Slaughter, C.; Raducha, M.; Harris, H. Isolation and characterization of a cDNA encoding a human liver/bone/kidney-type alkaline phosphatase. PNAS 1986, 83, 7182-7186.
27. Swallow, D. M. Povey, S. Parkar, M. Andrews, P. W. Harris, H. Pym, B. Goodfellow, P. Mapping of the gene coding for the human liver/bone/kidney isozyme of alkaline phosphatase to chromosome 1. Annals of Human Genetics 1986, 50: 229-35
28. Mornet, E. Stura, E. Lia-Baldini, A. S. Stigbrand, T. Ménez, A. Le Du, M. H. Structural evidence for a functional role of human tissue nonspecific alkaline phosphatase in bone mineralization. J Biol Chem. 2001, 276: 31171-8
29. Ferguson, M. A. Hart, G. W. Kinoshita, T. Glycosylphosphatidylinositol Anchors. Essentials of Glycobiology [Internet]. 3rd edition. Cold Spring Harbor (N.Y.): 2015-2017, Chapter 12
30. Whyte, M. P. Hypophosphatasia and the role of alkaline phosphatase in skeletal mineralization. Endocr Rev. 1994, 15: 439-61
31. Spentchian, M. Brun-Heath, I. Taillandier, A. Fauvert, D. Serre, J. L. Simon-Bouy, B. Carvalho, F. Grochova, I. Mehta, S. G. Müllner, G. Oberstein, S. L. Ogur, G. Sharif, S. Mornet, E. Characterization of missense mutations and large deletions in the ALPL gene by sequencing and quantitative multiplex PCR of short fragments. Genet Test. 2006, 10: 252-7
32. Cohen, P. A.; Strauss, J. V.; Rooney, A. D.; Sharma, M.; Tosca, N. Controlled hydroxyapatite biomineralization in a ˜810 million-year-old unicellular eukaryote. Sci. Adv. 2017, 3, 1-8.
33. Brundavanam, R. K.; Poinern, G. E. J.; Fawcett, D.; Modelling the Crystal Structure of a 30 nm Sized Particle based Hydroxyapatite Powder Synthesized under the Influence of Ultrasound Irradiation from X-ray Powder Diffraction Data. Am. J Mater. Sci. 2013, 3, 84-90.
34. Boonrungsiman, S.; Gentleman, E.; Carzaniga, R.; Evans, N. D.; McComb, D. W.; Porter, A. E.; Stevens, M. M. The role of intracellular calcium phosphate in osteoblast-mediated bone apatite formation. PNAS 2012, 109, 14170-14175.
35. Lotsari, A.; Rajasekharan, A. K.; Halvarsson, M.; Andersson, M. Transformation of amorphous calcium phosphate to bone-like apatite. Nat. Commun. 2018, 9: 4170.
36. Kostov-Kytin, V. V.; Dyulgerova, E.; Ilievac, R.; Petkova, V. Powder X-ray diffraction studies of hydroxyapatite and β-TCP mixtures processed by high energy dry milling. Ceram. Int. 2018, 44, 8664-8671.
37. Leadbeater, B. S. C.; Riding, R. Biomineralization in Lower Plants and Animals; Clarendon Press: Oxford, U K, 1986.
38. Bentov, S.; Aflalo, E. D.; Tynyakov, J.; Glazer, L.; Sagi, A. Calcium phosphate mineralization is widely applied in crustacean mandibles. Sci. Rep. 2016, 6, 22118.
39. Porter, S. M. Seawater chemistry and early carbonate biomineralization. Science 2007, 316, 1302.
40. Shen, W.; Wei, Y.; Dauk, M.; Tan, Y.; Taylor, D. C.; Selvaraj, G.; Zou, J. Involvement of a glycerol-3-phosphate dehydrogenase in modulating the NADH/NAD+ ratio provides evidence of a mitochondrial glycerol-3-phosphate shuttle in Arabidopsis. Plant Cell 2006, 18, 422-441.
41. Pettigrew, D. W.; Ma, D.; Conrad, C. A.; Johnsonn, J. R. Escherichia coli Glycerol Kinase. J. Biol. Chem. 1988, 263, 135-139.
42. Hayashi, S.; Koch, J. P.; Lin, C. C. Active transport of L-a-glycerophosphate in Escherichia coli. J. Biol. Chem. 1964, 239, 3098-3105.
43. Robison, R.; Soames, K. M. The possible significance of hexosephosphoric esters in ossification: Part II. The phosphoric esterase of ossifying cartilage. Biochem. J. 1924, 18, 740-754.
44. Siller, A. F.; Whyte, M. P. Alkaline phosphatase: Discovery and naming of our favorite enzyme. JBMR 2017, 33, 362-364.
45. Hunter, L. W.; Shiekh, F. A.; Pisimisis, G. T.; Kim, S. H.; Edeh, S. N.; Miller, V. M.; Lieske, J. C. Key role of alkaline phosphatase for development of human-derived nanoparticles in vitro. Acta Biomater. 2011, 7, 1339-1345.
46. Haugen, H. J. Monjo, M. Rubert, M. Verket, M. Lyngstadaas, S. P. Ellingsen, J. E. Hans Jacob Rønold, H. J. Wohlfahrt, J. C. Porous ceramic titanium dioxide scaffolds promote bone formation in rabbit peri-implant cortical defect model. Acta Biomaterialia 2013, 9: 5390-99.

1. (canceled)

2. A method of making HAP comprising:
contacting cells with calcium and an acyclic alkane phosphoester salt or inorganic phosphate salt, wherein the cells do not express an alkaline phosphatase, and contacting the cells with an alkaline phosphatase, wherein the cells produce HAP.

3. (canceled)

4. (canceled)

5. The method of claim 2, wherein the cells are contacted with 0.05-0.5 U/ml alkaline phosphatase.

6. The method of claim 2, wherein the alkaline phosphatase is selected from the group consisting of tissue nonspecific alkaline phosphatase (ALPL), calf intestinal alkaline phosphatase (CIP), shrimp hepatopancreas alkaline phosphatase (SAP), and asfotase alfa recombinant alkaline phosphatase.

7. The method of claim 2, wherein the acyclic alkane phosphoester salt is selected from disodium β-glycerophosphate, disodium α-glycerophosphate, phosphoenolpyruvate sodium, disodium dihydroxyacetone phosphate, or dilithium dihydroxyacetone phosphate.

8. The method of claim 7, wherein the acyclic alkane phosphoester salt is disodium α-glycerophosphate.

9. The method of claim 2, wherein the cells are contacted with 0.5-5 mM acyclic alkane phosphoester salt and 0.001-1M inorganic phosphate salts.

10. (canceled)

11. The method of claim 2, wherein the cells are contacted with 0.2-2 mM calcium.

12. The method of claim 2, wherein the HAP produced by the method has the chemical formula of Ca₁₀(PO₄)₆(OH)₂.

13. (canceled)

14. (canceled)

15. (canceled)

16. The method of claim 2, wherein the cells are selected from MG63, Saos-2, hFOB 1.19, HS-5, HEK-293, RD, HCN-2, MDA-MB-231, MCF-7, SW620, Colo205, Colo32DM, Du145, LNCap FGC, PC-3, HeLA, H₉₂₉, ARK, ARP1, CAG, JJN3, OPM2, RPMI8226, U266, HL-60, K-562, THP1, U937, C2C12, and NIH-3T3.

17. The method claim 2, further comprising harvesting the HAP produced by the cells.

18. (canceled)

19. The method of claim 2, further comprising incubating the cells with an object and allowing the HAP to collect on and/or coat the object.

20. A method of collecting the hydroxyapatite (HAP) made by the method of claim 2 comprising
a. fixing the cells with an aldehyde and collecting the fixed cells;
b. washing the fixed cells with a basic solution and collecting the pellet; and
c. extracting the pellet with acetone or chloroform.

21. The method of claim 20, wherein the aldehyde is a 10% buffered formalin solution.

22. The method of claim 2021, wherein the basic solution in step (b) is a 10% sodium hydroxide solution.

23. (canceled)

24. The method of claim 20, further comprising washing the extract with ethanol.

25. (canceled)

26. HAP made by the method of claim 20.

27. (canceled)

28. The HAP of claim 26, wherein the HAP forms crystallite particles between 0.1 nm and 40 nm in size.

29. A method of using the HAP produced by the method of claim 2, comprising contacting an object with the HAP.

30. The method of claim 29, wherein the object comprises collagen, a pharmaceutical agent, a medical device, a scaffold or an implant.

31. (canceled)

32. (canceled)

33. A method of measuring organic phosphates in a sample from a subject, comprising:
a. obtaining a sample from the subject;
b. preparing a supernatant from the sample;
c. heat inactivating a portion of the supernatant of step (b);
d. incubating the supernatant of step (b) and the product of step (c) with alkaline phosphatase for at least 2 hours;
e. performing a phosphorus detection assay and comparing the treated supernatant of step (b) with the heat inactivated supernatant of step (c), wherein the difference equals the quantity of organic phosphates in the sample.

34. (canceled)

35. A method of measuring glycerophosphates in a sample from a subject comprising:
a. obtaining a sample from the subject;
b. preparing a supernatant from the sample;
c. incubating the supernatant with a detectable substrate and a glycerophosphate oxidase;
d. measuring the detectable substrate of the reaction of step (c).

36.-40. (canceled)
